(19) United States
(12) Patent Application Publication
(10) Pub. No.: US 2018/0093943 A1
(43) Pub. Date: Apr. 5, 2018

(54) Processes and Intermediates for Preparing a Macrocyclic Protease Inhibitor of HCV

(71) Applicant: Janssen Pharmaceuticals, Inc., Titusville, NJ (US)

(72) Inventors: Dominique Paul Michel Depré, Beerse (BE); Dominic John Ormerod, Beerse (BE); Andras Horvath, Beerse (BE); Thomas Shaw Moody, Craigavon (IE); Maude Brossat, Craigavon (IE); Olivier Riant, Louvain-la-Neuve (BE); Nicolas Vriamont, Louvain-la-Neuve (BE); Sébastien Francois Emmanuel Lemaire, Belgium (BE); Sébastien Nicolas J. Hermant, Beerse (BE)

(73) Assignee: Janssen Pharmaceuticals, Inc., Titusville, NJ (US)

(21) Appl. No.: 15/559,910

(22) PCT Filed: Mar. 25, 2016
(86) PCT No.: PCT/IB2016/051720

§ 371 Date: Sep. 20, 2017

(30) Foreign Application Priority Data
Mar. 27, 2015 (EP) 15161431.0

(51) Int. Cl. C07C 235/82; C07C 67/31; C07C 231/02; C07C 231/12; C07D 417/04; C07D 417/14

(52) U.S. Cl. CPC C07C 235/82; C07C 67/31; C07C 231/02; C07C 69/757; C07D 417/04; C07D 417/14; C07C 231/12

(57) ABSTRACT

Disclosed is a process for the preparation of certain intermediates, e.g. those in the scheme below: which intermediates and processes are useful in the preparation of the macrocyclic HVC inhibitor Simeprevir.

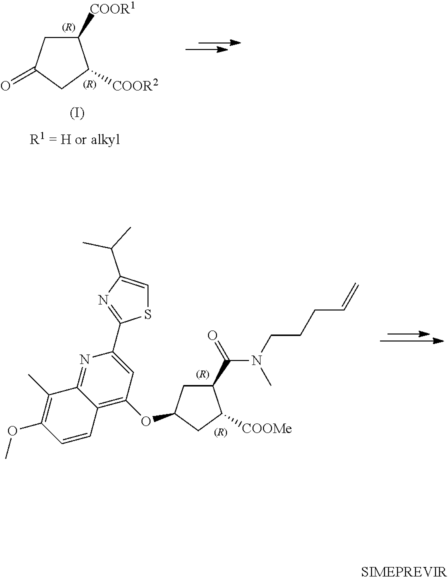

### FIELD OF THE INVENTION

The present invention relates to synthesis procedures and synthesis intermediates of a macrocyclic protease inhibitor of the hepatitis C virus (HCV), namely, simeprevir. Hence, there is also provided processes ultimately for the preparation of simeprevir.

### BACKGROUND OF THE INVENTION

The Hepatitis C Virus (HCV) is the leading cause of chronic hepatitis, which can progress to liver fibrosis leading to cirrhosis, end-stage liver disease, and HCC (hepatocellular carcinoma), making it the leading cause of liver transplantations.

Simeprevir is now approved (in at least the US, Europe and Japan) for the treatment of certain types of HCV. It is approved for use in combination with other agents as for example per the Summary of Product Characteristics at EMA.

Replication of the genome of HCV is mediated by a number of enzymes, amongst which is HCV NS3 serine protease and its associated cofactor, NS4A. Simeprevir works by inhibiting this enzyme and in the clinic it has shown pronounced activity against HCV and an attractive pharmacokinetic profile, leading to its approval. It has the following structure:

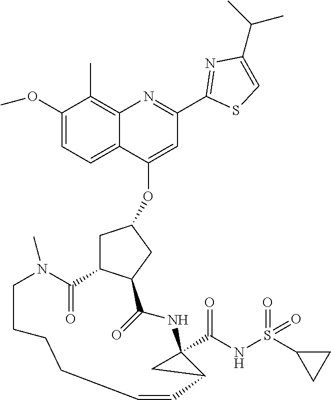 [SMILES: COc1ccc2c(O[C@@H]3C[C@H]4C(=O)N[C@]5(C(=O)NS(=O)(=O)C6CC6)C[C@H]5/C=C\CCCCN(C)C(=O)[C@@H]4C3)cc(-c3nc(C(C)C)cs3)nc2c1C]

Several international patent applications including WO 2007/014926, WO 2008/092955, WO 2010/072742, WO 2011/113859, WO 2013/041655 and WO 2013/061285 disclose a number of different processes to obtain simeprevir via a number of different intermediates (some of which are themselves new). Within these patent documents, there are also other references concerning related processes or certain intermediates used in those processes.

The references themselves may be referred to for full details of the processes, but it is clear that certain intermediates, in particular I mentioned below, may be a key intermediate in the synthesis of Simeprevir (Scheme 1).

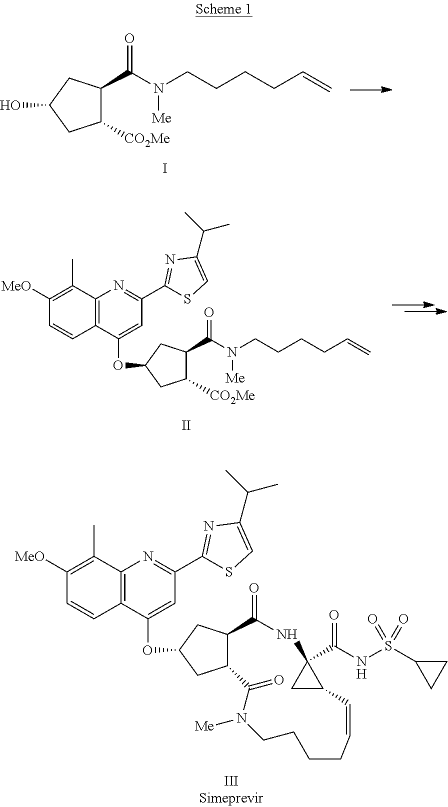

A first synthesis of I has been described in the prior art (Scheme 2), where starts from racemic keto-diacid IV and goes through the formation of the racemic lactone-acid VI, which is resolved by crystallization of diastereoisomeric salts with cinchonidine VII. This compound is then allowed to react with the secondary amine VIII to afford the lactone-amide IX, which is further converted into the compound I with methanol (further details are described in inter alia WO 2010/072742).

Alternatives/improvements to such processes are described in inter alia WO 2013/041655.

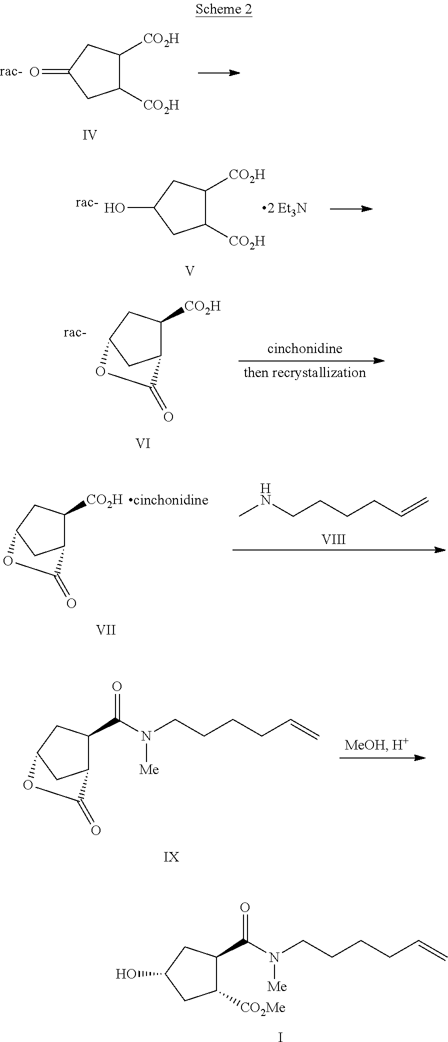

Further syntheses, for instance those described in WO 2011/113859, for preparing the intermediate I are also know. With reference to Scheme 3, it can be seen that resolution of keto-diacid IV may be performed earlier in the synthesis, by crystallization of diastereoisomeric salts X or XI with either brucine or (1R,2S)-ephedrine, respectively. The enantio-enriched keto-diacid XII thereby obtained was further converted into I (Scheme 3).

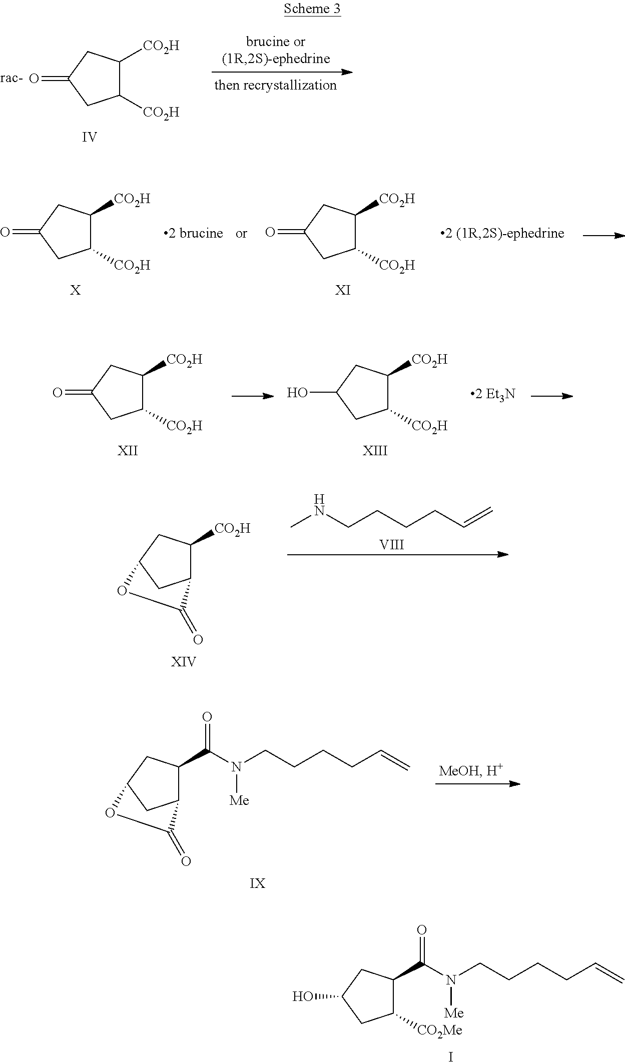

In addition to the above-mentioned processes there are also several other known processes to prepare Simeprevir. However, as indicated above, compound I above may be used as a key intermediate. Although processes for preparing I exist, there is a need for alternative and/or improved processes.

Process for preparing certain intermediates using enzymatic conditions are described in journal article by Hans Hilpert et al, J. of Med. Chem. Vol 56(23), 2013, pp 9789-9801 and by Rosenquist et al, Acta Chem. Scand., Vol 46(11), 1992, p1127-1129.

For instance, there may now be provided an alternative and/or improved process to prepare intermediates I and H (as depicted in Scheme 1) and therefore an alternative and/or improved process for preparing Simeprevir (see III in Scheme 1).

### DESCRIPTION OF THE INVENTION

In one aspect, the present invention relates to a process for preparing a compound of formula (I)

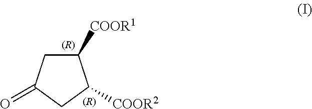

wherein
R¹ represents hydrogen or an alkyl group (for example a C₁₋₆ alkyl group, especially methyl);
R² represents an alkyl group (for example a C₁₋₆ alkyl group, especially methyl); the two chiral centres are of an (R) configuration (thereby representing an enantioenriched form);
which comprises selective hydrolysis of a (trans-racemic) compound of formula (II)

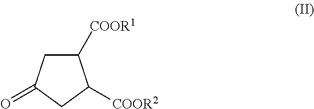

but in which
R¹ and R² independently represent an alkyl group (for example a C₁₋₆ alkyl group, especially methyl),
which process may be referred to herein as a process of the invention.

For the avoidance of doubt, the compound of formula (II) starting material is racemic and the respective groups —COOR¹ and —COOR² are in a trans-relationship (there is no “cis”-compound).

The selective hydrolysis is in fact a kinetic resolution, conditions for which are discussed hereinafter. Hence, only one of the carboxylic ester moieties is hydrolysed, preferably (as can be seen from the embodiment depicted above), the hydrolysis involves a compound of formula (II) in which R¹ is alkyl being converted to a compound of formula (I) in which R¹ is hydrogen, as per the following Scheme A:

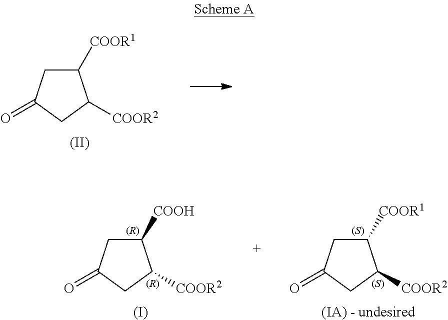

The selective hydrolysis (see conditions below) favours one of the two enantiomers of the racemic mixture of (II), which enantiomers are differentiated in the hydrolysis step (hence “selective”). Hence, the compound of formula (I) in which R¹ is H, where each of the two chiral centres has a (R)-configuration, is the desired product formed. As a consequence, the process will result in the undesired enantiomer that is non-hydrolysed, i.e. that of formula (IA) in which R¹ remains an alkyl group and each chiral centre has a (S)-configuration. As explained below, the undesired compound/enantiomer may easily be removed.

Additionally, there may (either as a result of a minor side-reaction or through use of different selective hydrolysis conditions) be a process in which the different products are produced in accordance with Scheme B:

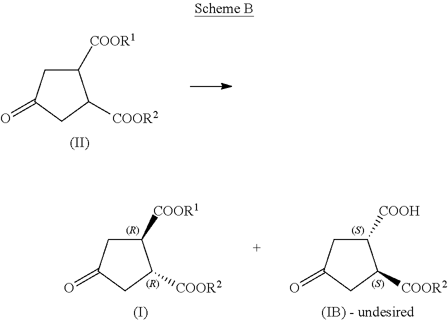

In the above Scheme B, it is the enantiomer with the (S,S) configuration that is selectively hydrolysed (to yield compound (IB)) and hence here the compound of formula (I) is that in which the R¹ group represents alkyl (and the two chiral centres are in the (R)-configuration). Again, it is compound (I) which is desired (in the downstream synthesis of Simeprevir) and, like for the case above, the undesired compound/enantiomer of formula (IB) may easily be removed.

Advantageously, a compound of formula (I), i.e. in which each chiral centre is of (R)-configuration, in which R¹ represents H or in which R¹ represents alkyl may be converted to Simeprevir, as detailed below. Although, advantageously a compound of formula (I) in which R¹ is H is employed, this compound, if desired, can also be converted to a compound of formula (I) in which R¹ is alkyl (for example under standard esterification conditions, employing the relevant alkyl alcohol e.g. under acidic H⁺ conditions). Hence, there is provided a use of a compound of formula (I) in which R¹ is H or alkyl (in particular in which R¹ is H) in the preparation of Simeprevir (or an intermediate thereto, such as a key one depicted in the scheme below):

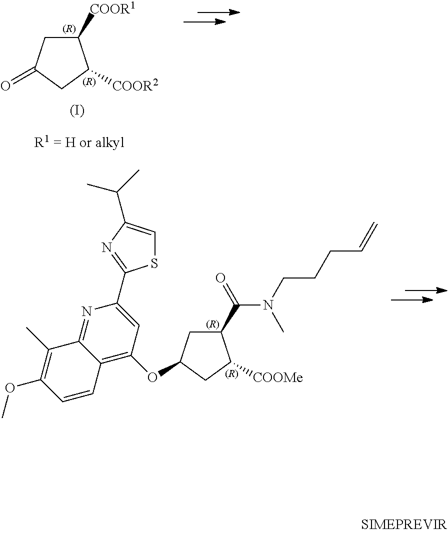

First, the racemic compound (II) may be prepared from the corresponding di-acid by reaction with the desired alkyl alcohol (e.g. methanol for the conversion to methyl esters) optionally in the presence of acid (for example an appropriate source of H⁺, for example concentrated sulfuric acid). Although such reaction may be performed in the presence of the alkyl alcohol as solvent, optionally a further solvent may be added, for example toluene. Such reaction mixture may be heated at reflux. The desired product (compound of formula (II)) may be extracted using standard procedures.

In order to perform the selective hydrolysis in the step above (i.e. compound (II) to compound (I)), the racemic compound of formula (II) is allowed to undergo a hydrolysis reaction in the presence of a suitable catalytic enzyme. Suitable enzymes include those that allow selective hydrolysis to form a —COOH group with a (R)-configuration at the relevant chiral centre (although a minor amount of the (S)-configuration may be obtained, the desired product was in any event obtained in enantioenriched form; such enantioenrichment may further be enhanced by purification techniques as described hereinbelow, for example, by purification techniques to remove the undesired enantiomer). The enzyme is preferably of the hydrolase class, such as, but not limited to, lipase, esterase, protease, aminoacylase, especially a lipase enzyme (for example as described hereinafter).

Advantageously, the use of an enzymatic selective hydrolysis step allows manipulation of the hydrolysis to favour the formation of whichever product is desired—hence conditions may be changed (e.g. as described hereinbelow) to favour hydrolysis of the (R,R)-enantiomer (hence producing a compound of formula (I) in which R¹ is H) or to favour hydrolysis of the (S,S)-enantiomer (hence producing a compound of formula (I) in which R¹ is alkyl); further the undesired product (diester or mono-acid, respectively) can be removed (e.g. as indicated below, through the work-up).

The most preferred lipase enzymes, for example to directly obtain the compound of formula (I) i.e. the (R,R)-configuration in which R¹ represents hydrogen are:

Lipase B from Candida Antartica (CAL-B)

Immobilized lipase A from Candida Antartica B (immobilized CAL-B)

The most preferred lipase enzymes, for example to obtain the compound corresponding to that of formula (I) in which R¹ represents hydrogen but which has an (S,S)-configuration, and thereby also producing a compound of formula (I) in which R¹ represents alkyl (corresponding to the alkyl starting material) are:

Protease from Aspergillus Melleus

Aminoacylase from Aspergillus sp.

The relevant hydrolysase enzymes (mentioned herein) can be obtained from Almac or from another suitable supplier.

It is preferred that the reaction is optionally performed in the presence of a suitable solvent, for example an organic solvent (e.g. an apolar solvent, an aprotic solvent or a polar aprotic solvent, such as toluene, ether, 1,2-dimethoxyethane, THF, acetone, 1,4-dioxane, hexane, methyl ethyl ketone (MEK) or the like).

It is preferred that the racemic starting material (compound of formula II, preferably the keto-dimethyl ester) is shaken or stirred in solvent (for example at room temperature, for a period of time greater than 1 hour, e.g. greater than 4 hours, overnight) together with a solution of enzyme (preferably in buffer). The buffer is preferably a phosphate buffer (e.g. 0.1M phosphate buffer pH 7). The pH may be adjusted as appropriate, e.g. depending on the enzyme that is employed as every enzyme has its own optimal pH for activity and selectivity. The pH can then be further adjusted for work-up purposes: for instance the reaction mixture may be acidified to low pH (e.g. pH 1-5) by use of concentrated HCl before removal of the enzyme and extraction of the desired products.

Specific conditions for specific enzymes may be described herein in the examples. The skilled person may be able to adapt the conditions as desired/appropriate.

The process of the invention produces enantioenriched products, by which we mean the products produced have an enantiomeric excess of greater than 20%, preferably greater than 40%, such as more than 60% and especially greater than 80% enantiomeric excess. The enantioenriched products may even be greater than 90% (for example, they may consist essentially of a single enantiomer, by which we mean that the ee may be 95% or higher, e.g. above 98% or about 100%). Such enantioenrichment (or ees) may be obtained directly, or through further purification techniques that are known to those skilled in the art. For instance, the process of the invention may produce a compound of formula (I) in which R¹ represents H, wherein such a product is enantioenriched.

Hence, in an embodiment of the invention, there is provided a compound of formula (I) in which R¹ represents H (and R² is as defined herein, preferably methyl), i.e.:

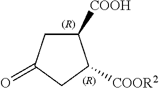

wherein the product is enantioenriched (e.g. greater than 20% ee, and, especially greater than 80% ee, for example about 100% ee). Such product may easily be isolated from the process reaction of the invention.

In the instance where such a product (compound of formula (I) in which R¹ is H) is formed, then in the reaction mixture of the process of the invention, there will potentially also remain unreacted starting material (compound of formula (II)) and the non-hydrolysed enantiomer (compound of formula (I) but in which the configuration of the chiral centres is (S,S)-configuration, and R¹ does not represent H, i.e. both R¹ and R² independently represent alkyl, such as methyl)—such compounds may advantageously be removed in the work up or extraction process. For instance, the keto-mono-carboxylic acid and the keto-diesters have different properties enabling a partitioning step between an organic layer and an aqueous layer by control of the pH of the aqueous layer. Hence the mono-acid functionality versus the di-ester functionality may be exploited in order to easily separate the products.

For instance, the partitioning step, when the mono-acid, mono-ester product is the desired one may be performed by allowing the reaction mixture to mix with water and an organic solvent (immiscible with water) and raising the pH and/or maintaining the pH ≥7 (e.g. by adding base, such as sodium hydroxide) thereby allowing the desired mono-acid (as its carboxylate salt) to go into the alkaline aqueous layer. In this instance, once the aqueous layer is separated, the pH thereof can then be lowered (e.g. to around pH 2), and the desired product (in this case the mono-acid, i.e. compound of formula (I) in which R¹ is H) may be extracted with any suitable organic solvent (e.g. ethyl acetate).

Equally, if the selective hydrolysis reaction is performed with an enzyme that selectively produces a mono-acid of the corresponding (S,S)-enantiomer, then the desired diester (i.e. compound of formula (I) in which R¹ is alkyl) may be separated (from the mono-acid) by raising the pH and/or maintaining the pH ≥7 and allowing the diester to remain in the organic layer (the mono-acid being in the aqueous layer as its carboxylate salt). Extraction may then be performed under standard conditions.

In this respect, in an embodiment of the invention, there is also provided a compound of formula (I) in which R¹ represents alkyl (preferably methyl) (and R² is as defined herein, preferably methyl), i.e.:

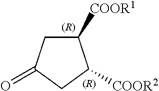

in which R¹ and R² independently represent alkyl (e.g. methyl, so forming a dimethyl-ester), and wherein the product is enantioenriched (e.g. greater than 20% ee, and, especially greater than 80% ee, for example about 100% ee). Such product may easily be isolated from the process reaction of the invention.

In the above-mentioned ways, compounds produced by means of the process of the invention may be purified and therefore substantially isolated from other undesired by-products or from unreacted starting material. Other standard purification or isolation techniques may also be employed.

The desired product of formula (I) formed has the advantage that the preceding racemate is (i) resolved (affording the desired enantiomer) and (ii) one of the carboxylic ester moieties is hydrolysed selectively (which is desirable for downstream steps in synthesizing Simeprevir). The resolution and the differentiation of the two carboxylic ester groups in one step may increase efficiency of the process.

As indicated above, compounds of formula (I) are key intermediates to Simeprevir via certain other key intermediates.

Hence, compounds of formula (I) (in which R¹ is H or alkyl) can further be converted as follows (which processes may also be embodiments of the invention):
(i) Compounds of formula (I) in which R¹ is H may be converted to compounds of formula (I) in which R¹ is alkyl, under standard esterification conditions;
(ii) Compounds of formula (I) in which R¹ is H may undergo a standard amide-coupling reaction in accordance with the following scheme:

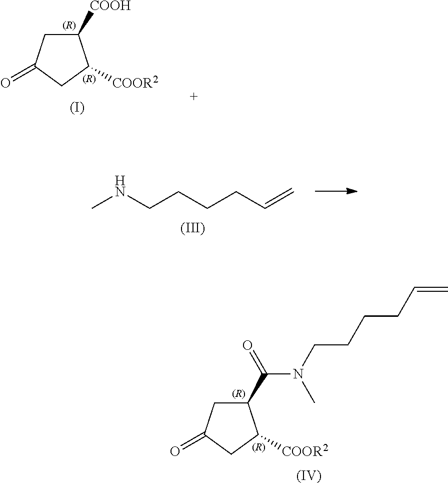

Such reaction may be performed under standard conditions, for example amide-coupling reaction conditions (as may be described hereinafter);
(iii) Compounds of formula (I) may also undergo a diastereoselective reduction reaction in accordance with the following scheme:

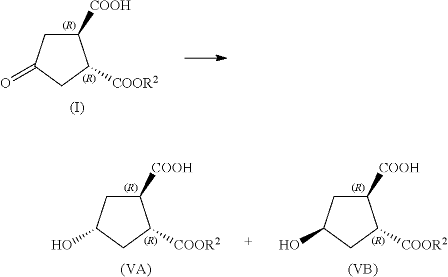

Hence, this advantageously generates a third stereogenic/chiral centre, with the major product obtained (i.e. (R)-alcohol diastereoisomer or the (S)-alcohol diastereoisomer) being dependent on the reagents and conditions used; such conditions are discussed in more detail below. Where a mixture of such enantiomers is obtained (i.e. of (VA) and (VB)), such diastereoisomers may be separated under standard known conditions (for example, a chromatographic separation);
(iv) Compounds of formula (IV) may also undergo a diastereoselective reduction reaction in accordance with the following scheme:

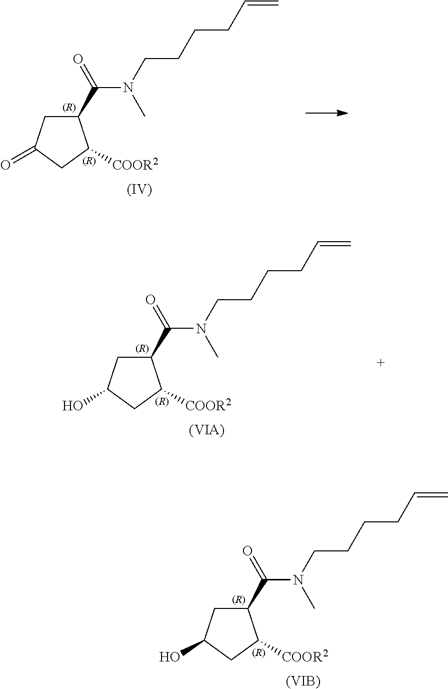

As for (iii) above, this advantageously generates a third stereogenic/chiral centre, with the major product obtained (i.e compound of formula (VIA) or (VIB)) being dependent on the reagents and conditions used; such conditions are discussed in more detail below. Where a mixture of such diastereoisomers is obtained (i.e. of (VIA) and (VIB)), such diastereoisomers may be separated under standard known conditions (for example, a chromatographic separation);
(v) Compounds of formula (VA) and (VB) may be converted to compounds of formula (VIA) and (VIB), respectively, for example under the conditions mentioned herein at (ii) above, i.e. an amide-coupling reaction in the presence of an amine of formula (III) and under the appropriate reaction conditions;
(vi) Compounds of formula (I) in which R¹ and R² both represent alkyl may also be converted, by a ketone reduction reaction, in accordance with the following scheme:

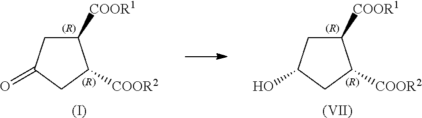

Under reduction reaction conditions (see e.g. (iii) above), which advantageously introduces a further stereogenic/chiral centre;
(vii) Compounds of formula (VII) may undergo a selective hydrolysis reaction to form compounds of formula (VA) and (VB) (for conditions see e.g. conditions described for the main process of the invention, i.e. conversion of (II) to (I));
(viii) Compounds of formula (VIA) may be converted to compounds of formula (IX) by reaction with intermediate (VIII) in accordance with the scheme below:

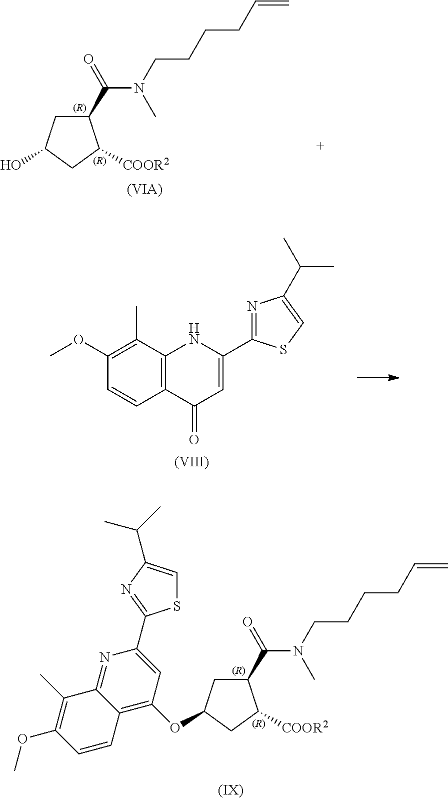

Such reaction may be performed under reaction conditions described in prior art documents such as those mentioned in the background (e.g. WO 2011/113859). Such aliphatic nucleophilic substitution (e.g. a so-called Mitsunobu reaction) occurs with inversion of configuration (SN2); hence why the configuration at the “OH” group inverts;
(ix) Compounds of formula (VIB) may also be converted to compounds of formula (IX) (by reaction with intermediate (VIIIA)) in accordance with the scheme below:

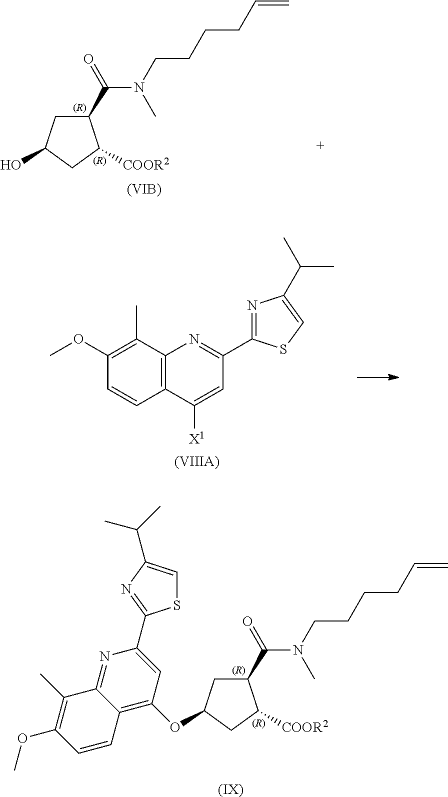

In this case, the aromatic nucleophilic substitution reaction proceeds with retention of configuration (SNAr); X¹ is a suitable leaving group (e.g. halo, such as chloro, bromo or iodo, a sulfonate group, e.g. —OSO₂X^{a} (where X^{a} may be optionally substituted alkyl or aryl, e.g. —CH₃, —CF₃ or —C₆H₄—CH₃) so forming e.g. mesylate, triflate or tosylate, or the like) and R² may be alkyl (as defined herein) or may be H in this case (so forming the corresponding carboxylic acid). Reaction conditions for such a conversion may be described hereinafter;
(x) Compounds of formula (VIIIA) may be prepared from compounds of formula (VIII) for example under standard procedures or as described hereinafter.

As stated above, such further conversion steps, i.e. processes described at (i) to (x) above may themselves also be embodiments of the invention, for example processes described at (iii) and (iv) above may also be embodiments of the invention. In particular as the conditions (see e.g. hereinbelow) may be manipulated to favour one of the two diastereoisomers (e.g. in (VA) or, preferably, (VB) in the case of (iii) and (VIA) or, preferably, (VIB) in the case of (iv)). There is therefore provided a process at described at (iii) or (iv) above to produce, preferably (VB) or (VIB), respectively.

For the reduction (e.g. diastereoselective reduction) reaction (see (iii), (iv) and (vi) above) possible conditions may be manipulated depending on which stereoisomer is desired (i.e. depending on whether an excess of the (R)- or (S)-alcohol is desired). It will therefore be understood that the order of reaction steps may be changed or reversed.

Standard reduction conditions may be employed, for example, using hydrogenation or a reducing agent such as lithium aluminium hydride or borohydride (e.g. sodium borohydride), or any suitable other hydride source, or hydrogen (for example as may be described in the examples hereinafter). The diastereoselectivity of the reduction can be controlled by the intramolecular complexation of the reductant with the carboxylic acid moiety of the relevant compound (e.g. (R,R)-XVIa as in Scheme 5 hereinafter; or compound of formula (I) e.g. as depicted in (iii) above), or controlled by the environment brought by an enzyme or an organometallic complex. Hence, advantageously, the reducing conditions may be manipulated to produce one or the other possible diastereoisomeric product (for example a reducing agent such as borohyride may advantageously produce Compound (VB) or (VIB) as the major product; in order to obtain (VA) or (VIA) other conditions may be employed, for example certain enzymatic conditions that may be manipulated to obtain either of the two diastereomers (VA) or (VB), or, (VIA) or (VIB), respectively for processes (iii) and (iv), or, alternatively reductions with silanes may be employed and may also be manipulated to form either one of the diastereoisomers, for example as described hereinbefore, e.g. in the examples). Hence, in sub-embodiments of the invention there may be provided:
A reduction as described in (iii) above, wherein the conditions (e.g. reduction using borohydride as described herein) are adapted to provide a compound of formula (VB) (for example, where (VB) is the major product compared to (VA), for example in the ratios mentioned hereinafter in the examples; and where the relevant conditions are described)
A reduction as described in (iv) above, wherein the conditions (e.g. reduction using borohydride as described herein) are adapted to provide a compound of formula (VIB) (for example, where (VIB) is the major product compared to (VA), for example in the ratios mentioned hereinafter in the examples; and where the relevant conditions are described)
A reduction as described in (iii) or (iv), where the reduction is enzymatic (for example under the conditions described herein) thereby providing either:
   (a) a compound of formula (VA) (as the major product, compared to (VB)) or a compound of formula (VIA) (as the major product compared to (VIB)), respectively; or
   (b) a compound of formula (VB) (as the major product, compared to (VA)) or a compound of formula (VIB) (as the major product compared to (VIA)), respectively, as, in each, the conditions or the enzyme may be manipulated in order to obtain the desired product (in the ratio desired)
A reduction as described in (iii) or (iv), where the reduction is performed with a silane, formic acid, or a salt thereof (for example under the conditions described herein, e.g. possibly with a chiral ligand) thereby providing either compound (VA) or (VB) as a major product, or, (VIA) or (VIB) as a major product (depending on the specific conditions employed, e.g. as may be described hereinafter; for instance certain chiral ligands may influence the stereoselectivity, e.g. by producing the compounds in a certain diastereoisomeric ratio). Specific chiral ligands may be described in the examples.

In some cases, the reduction step may be catalyzed by an enzyme of the oxydoreductase class, especially a ketoreductase (KRED), a carbonyl reductase (CRED) or an alcohol dehydrogenase (ADH)—all those terms are considered to be synonyms in this document—in the presence of a cofactor, either nicotine adenine dinucleotide (NADH) or nicotine adenine dinucleotide phosphate (NADPH), added into the reaction mixture either as their reduced form (NADH, NADPH), or as their oxidized form (NAD⁺, NADP⁺). The final reductant can be an alcohol like, but not limited to, isopropanol which is oxidized into acetone; this oxidation is catalyzed by the same enzyme that the one used for the reduction of the ketone XVII(a) (of Scheme 5 depicted in the examples hereinafter) or compound of formula (IV) depicted above. Alternatively, the final reductant can be either glucose, lactic acid or a salt thereof, or formic acid or a salt thereof whose oxidation into gluconic acid, pyruvate or carbon dioxide is catalyzed by a second enzymatic system (glucose dehydrogenase (GDH), lactate dehydrogenase or formate dehydrogenase respectively). The diastereoselectivity of the reduction of the ketone XVII(a) (referred to as compound of formula (IV) above) is achieved by the chiral environment brought by the enzyme. Specific enzymes and conditions may be described in the examples hereinafter.

Alternatively, the reduction of the ketone XVII(a) (or compound of formula (IV) above) can be catalyzed by an organometallic complex with either a silane, formic acid or a salt thereof or hydrogen as final reductant; the diastereoselectivity of the reduction is achieved by the environment brought by the organometallic complex. Specific silanes (or formic acid) and specific chiral ligands (and conditions) may be described in the examples hereinafter.

In the main process of the invention, a selective hydrolysis step is discussed. It is understood that the order of reaction steps may be changed, and hence the selective hydrolysis may be performed as per (vii) above. Such conditions as those described hereinbefore may be employed or, such hydrolysis of either one or the other ester moiety (see also scheme 6 hereinafter; which step permits the differentiation of the two carboxylic esters of the molecule) may be controlled by either the proximity of the hydroxyl moiety in the molecule [see e.g. M. Honda et al Tetrahedron Let. 1981, 22, 2679] or by the presence of an enzyme of the hydrolase class (e.g. see above).

Other conversions may be performed in accordance with standard techniques and steps in the prior art, for instance, amide-forming reactions (in this instance, possible conditions and coupling reagents will be known to those skilled in the art), esterifications, nucleophilic substitutions reactions and aromatic nucleophilic substitution reactions.

In further aspects of the invention, there is provided:
A compound of formula (IV) in enantioenriched form;
A compound of formula (VA) (in which R² does not represent hydrogen) and (VB), each of which are in enantioenriched form;
A compound of formula (VIB) in enantioenriched form;
A compound of formula (VII) (in which R² does not represent hydrogen) in enantioenriched form
A use of certain compounds herein (including e.g. (I), (IV), (VA), (VB), (VIB) and (VII)) as intermediates in the preparation of further compounds (such as compounds (IX), (X) and/or (XI), i.e. Simeprevir)

Advantageously, the compound of formula (IX) is a key intermediate in the synthesis of Simeprevir. The following conversion steps may be performed:

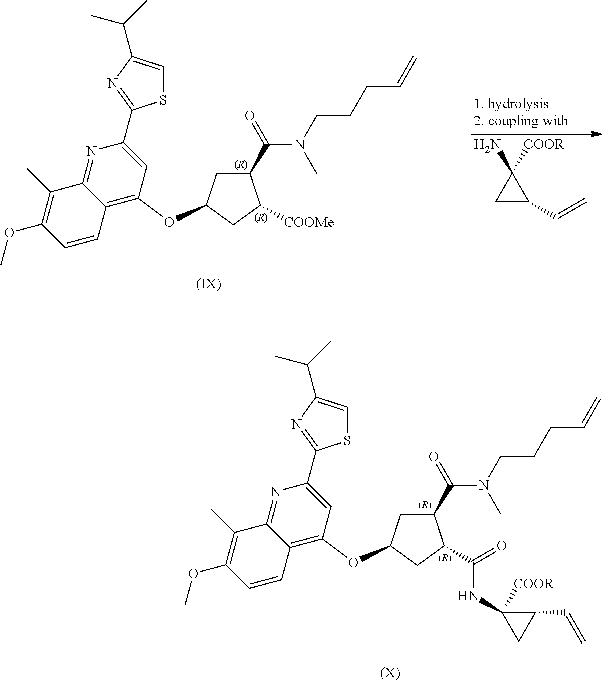

Compound (X) may then be further converted, for example as described in international patent applications WO 2007/014926, WO 2013/041655, WO 2013/061285 (or the references referred to therein). Hence, the following conversion may be performed:

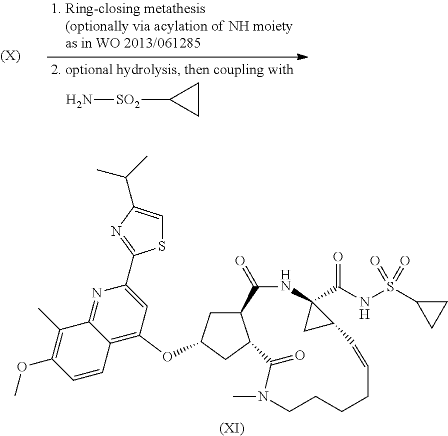

The compound of formula (XI) is Simeprevir, which may also be in the form of a salt (e.g. a sodium salt) and hence there is further provided a process of preparing, specifically, the sodium salt of the compound of formula (XI). There is then also provided a process for preparing a pharmaceutical formulation comprising (XI), or a salt thereof (e.g. a sodium salt thereof), wherein the compound of formula (XI) is prepared in accordance with procedures described herein (e.g. using the earlier processes, especially the conversion of the compound of formula (II) to (I) as described herein) and wherein the process for preparing the formulation comprises bringing into contact such a compound of formula (XI) (or salt thereof) with a pharmaceutically acceptable carrier, diluent and/or excipient.

### EXAMPLES

The following examples are intended to illustrate the present invention and should not be construed as a limitation of the scope of the present invention.

In the examples, the following Schemes 4 to 8 and Schemes 2 and 3 in the Background (and the associated compound numbering) may be referred to.

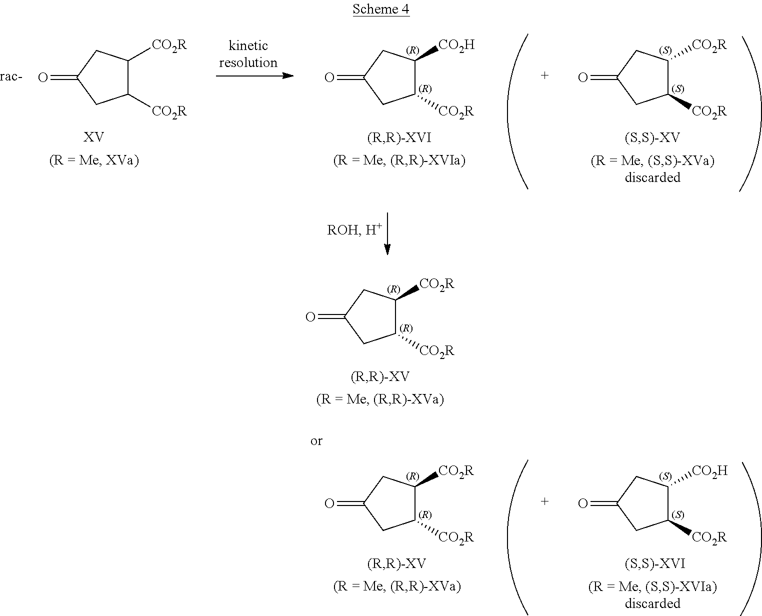

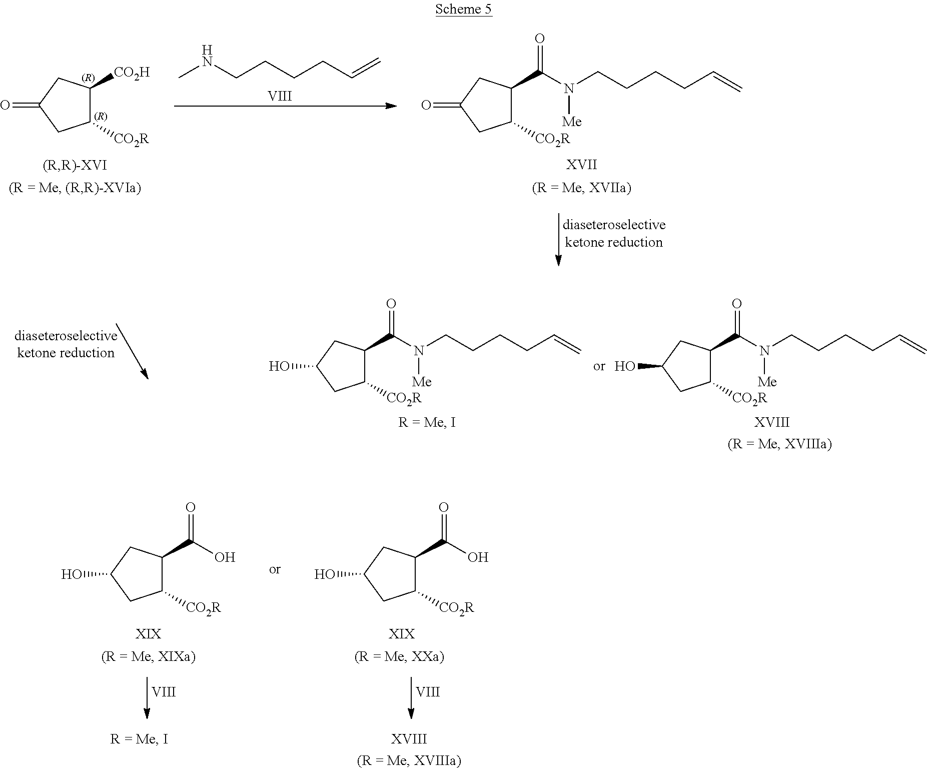

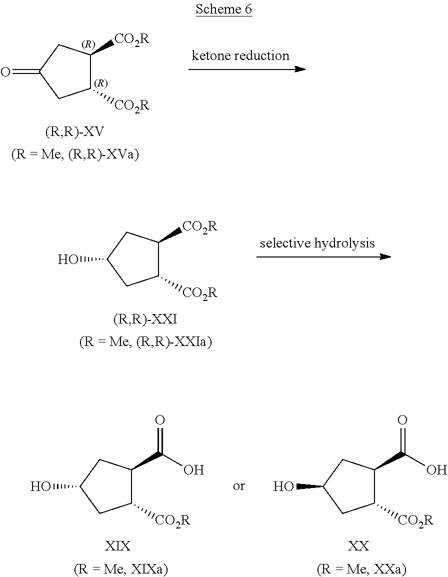

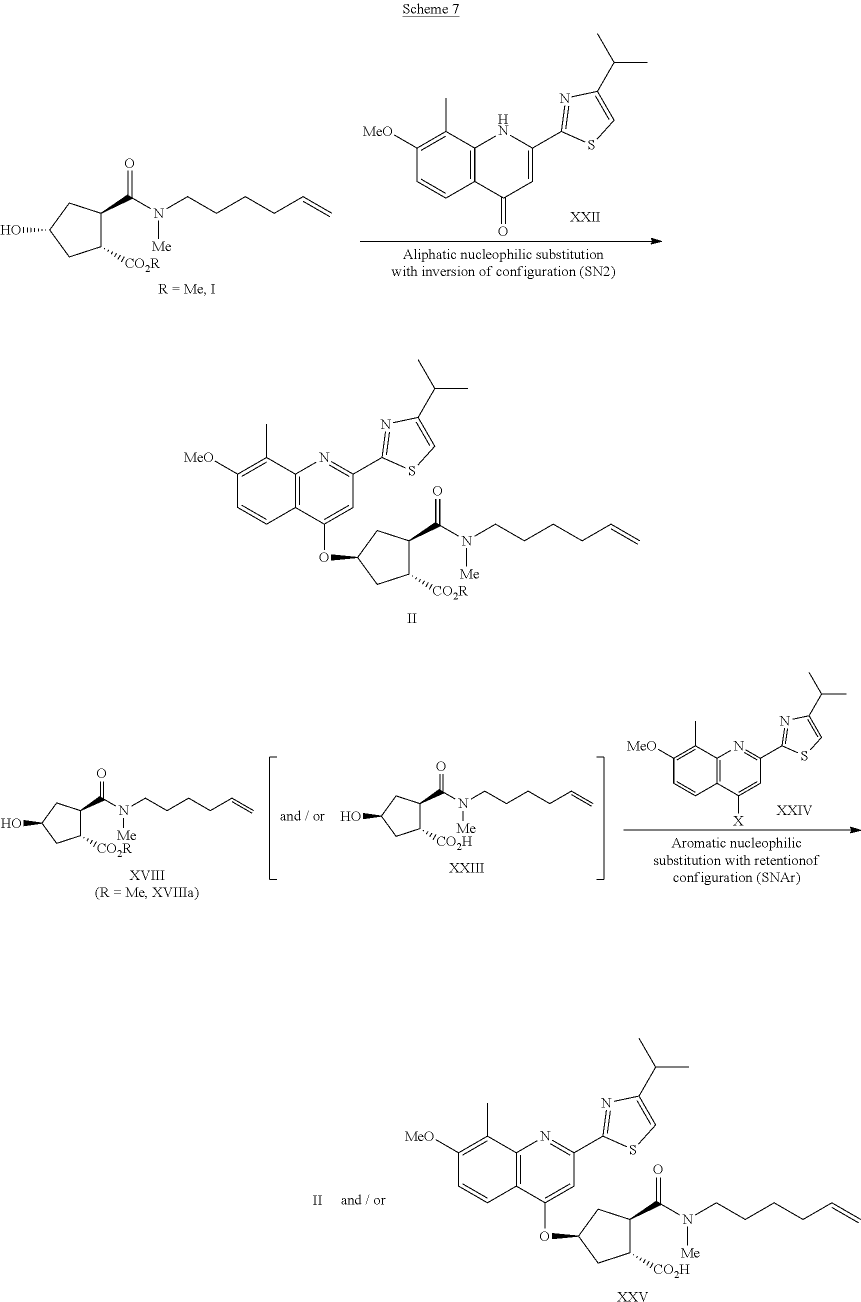

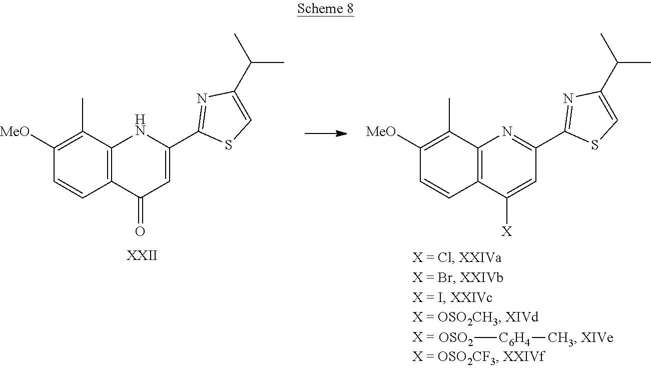

### EXAMPLES

### Example 1: Esterification of Compound IV into Compound XVa

8.4 ml (157 mmol) of concentrated sulfuric acid is added to a solution of 50 g (290 mmol) of compound IV, 290 ml of methanol, 435 ml of toluene and 5.3 ml (290 mmol) of water. The reaction mixture is refluxed and part of the solvent is distilled off until the internal temperature reaches 75° C. (370 ml solvent distilled). The reaction mixture is then cooled to 50° C. before addition of 58 ml of MeTHF and 290 ml of water. The biphasic mixture is allowed to settle and the two layers are separated. The organic layer is washed twice with 145 ml of water, dried over magnesium sulfate, filtered and concentrated under vacuum to dryness. 37.9 g of crude compound XVa is obtained as thick oil that solidifies on standing. Yield: 65%.

Characterization data was obtained for the desired product (CAS 28269-03-6), which was consistent with the literature.

### Example 2: Kinetic Resolution of Compound XVa (See Table of Screened Conditions Below)

A solution of 20 mg of compound XVa in 0.1 ml of organic solvent is shaken overnight at room temperature together with a solution of 10 mg of enzyme in 1 ml of 0.1M phosphate buffer pH 7.0. The reaction mixture is then acidified to pH 1 with concentrated HCl and the compounds of interest (compounds XVa and XVIa) are extracted with 0.3 ml of ethyl acetate. The organic solution is diluted and analyzed by chiral HPLC. The selectivity of the resolution is described by the dimentionless parameter “enantiomeric ratio” E introduced by Sih (C. J. Sih, S.-H. Wu, Topics Stereochem. 1989, 19, 63) and expressed by the following equation:

E=[ln [ee_{XVIa}·(1−ee_{XVa})/(ee_{XVIa}+ee_{XVa})]/[ln [ee_{XVIa}·(1+ee_{XVa})/(ee_{XVIa}+ee_{XVa})]

For the resolution to be of value, E is preferably at least 30.

| | | | | | Configuration |
|---|---|---|---|---|---|
| Example | | | | Configuration | of obtained |
| # | Enzyme | Solvent | E | of left XVa | XVIa |
| 2-1 | Protease from Aspergillus | toluene | 3.1 | (R,R) | (S,S) |
| | Melleus | | | | |
| 2-2 | Aminoacylase from | toluene | 25 | (R,R) | (S,S) |
| | Aspergillus sp. | | | | |
| 2-3 | Lipase B from Candida | toluene | 369 | (S,S) | (R,R) |
| | Antartica (CAL-B) | | | | |
| 2-4 | Immobilized lipase A | toluene | 243 | (S,S) | (R,R) |
| | from Candida Antartica | | | | |
| | B (immobilized CAL-B) | | | | |
| 2-5 | Esterase from pig liver | toluene | 3.3 | (R,R) | (S,S) |
| | (PLE) | | | | |
| 2-6 | Lipase B from Candida | None | 127 | (S,S) | (R,R) |
| | Antartica (CAL-B) | | | | |
| 2-7 | Lipase B from Candida | 1,2- | 141 | (S,S) | (R,R) |
| | Antartica (CAL-B) | dimethoxyethane | | | |
| 2-8 | Lipase B from Candida | THF | 74 | (S,S) | (R,R) |
| | Antartica (CAL-B) | | | | |
| 2-9 | Lipase B from Candida | Acetone | 106 | (S,S) | (R,R) |
| | Antartica (CAL-B) | | | | |
| 2-10 | Lipase B from Candida | 1,4-dioxane | 243 | (S,S) | (R,R) |
| | Antartica (CAL-B) | | | | |
| 2-11 | Lipase B from Candida | Hexane | 90 | (S,S) | (R,R) |
| | Antartica (CAL-B) | | | | |
| 2-12 | Lipase B from Candida | DMSO | 28 | (S,S) | (R,R) |
| | Antartica (CAL-B) | | | | |
| 2-13 | Lipase B from Candida | MEK | 116 | (S,S) | (R,R) |
| | Antartica (CAL-B) | | | | |

### Example 3: Kinetic Resolution of Compound XVa Towards Compound (R,R)-XVIa

10 g (50 mmol) of compound XVa was suspended at 15° C. in 200 ml of 0.1 M phosphate buffer pH 7.0. 1 g of immobilized CAL-B enzyme was added and the mixture was stirred for 6 hours at 15° C. with regular adjustment of the pH with 1 M sodium hydroxide solution. The pH was then adjusted to pH 8, the enzyme was filtered off and the unconverted diester compound (S,S)-XVa was extracted twice with 150 ml of ethyl acetate. The aqueous layer was acidified to pH 2 then extracted twice with 150 ml of ethyl acetate. The combined organic layers were dried and concentrated under vacuum to give 3.3 g of compound (R,R)-XVIa (yield: 36%, ee: 91%).

¹H NMR (360 MHz, CDCl₃) δ ppm 2.47-2.61 (m, 2H), 2.66-2.78 (m, 2H), 3.38-3.52 (m, 2H), 3.77 (s, 3H), 10.89 (br. s., 1H). ¹³C NMR (90 MHz, CDCl₃) δ ppm 40.65, 40.85, 43.26, 43.33, 52.58, 173.09, 177.74, 212.42. High resolution MS (EI, m/z): calcd for C₈H₁₀O₅ (M)⁺: 186.0528, found: 186.0531.

### Example 4: Kinetic Resolution of Compound XVa Towards Compound (R,R)-XVa

a) With Protease from Aspergillus Melleus

20 mg of compound XVa was suspended at 15° C. in 1 ml of 0.1 M phosphate buffer pH 7.0. 10 mg of Protease from Aspergillus Melleus was added and the mixture was stirred for 16 hours at room temperature. Conversion was 62%. The enantiomeric excess of the unconverted (R,R)-XVa was 52% and the enantiomeric excess of the compound (S,S)-XVIa was 32% (calculated E value of 3.1). The compound (S,S)-XVIa can be washed away with alkaline aqueous layer.

b) With Aminoacylase from Aspergillus sp

20 mg of compound XVa was suspended at 15° C. in 1 ml of 0.1 M phosphate buffer pH 7.0. 10 mg of Aminoacylase from Aspergillus sp was added and the mixture was stirred for 16 hours at room temperature. Conversion was 62%. The enantiomeric excess of the unconverted (R,R)-XVa was 65% and the enantiomeric excess of the compound (S,S)-XVIa was 86.5% (calculated E value of 12). The compound (S,S)-XVIa can be washed away with alkaline aqueous layer.

### Example 5: Esterification of Compound (R,R)-XVIa into Compound (R,R)-XVa

Compound (R,R)-XVa was obtained in 85% yield by refluxing compound (R,R)-XVIa in methanol in the presence of sulfuric acid following the procedure described in the example 1 for the esterification of the compound IV.

Characterization data (CAS 35079-19-7) fits with literature (eg Johansson, P.-O. et al Bioorganic & Medicinal Chemistry 2006, 14, 5136).

### Example 6: Amide Coupling from Compound (R,R)-XVIa to Compound XVIIa

### a) With EEDQ:

1 g of compound (R,R)-XVIa, xx g of compound VIII and xx g of EEDQ were dissolved in THF and stirred until complete conversion (ca overnight). 1.24 g of compound XVIIa was obtained after aqueous workup and purification by flash chromatography. Yield: 81%.

### b) With CDI:

A solution of 5 g of compound (R,R)-XVIa in MeTHF was added to a suspension of 1.3 equiv of CDI in MeTHF at 15-25° C. after complete conversion, 1.1 equiv of compound VIII was added and the reaction mixture was stirred until complete conversion. After aqueous washings (1M HCl then saturated sodium bicarbonate), the reaction mixture was concentrated under vacuum to deliver 6.45 g of compound XVIIa. Yield: 77%. Colorless liquid. [α]²⁵_{D}: −80.5 (c=1, MeOH). ¹H NMR (400 MHz, CDCl₃—1/1 mixture of two rotamers) 6 ppm 1.32-1.48 (m, 2H), 1.49-1.70 (m, 2H), 2.02-2.15 (m, 2H), 2.39-2.60 (m, 3H), 2.65-2.78 (m, 1H), 2.96 (s, 1.5H), 3.09 (s, 1.5H), 3.24-3.38 (m, 1H), 3.40-3.65 (m, 3H), 3.72 (s, 3H), 4.90-5.07 (m, 2H), 5.70-5.85 (m, 1H). ¹³C NMR (101 MHz, CDCl₃—1/1 mixture of two rotamers) 6 ppm 25.41, 25.48, 26.08, 27.71, 32.87, 32.96, 33.62, 34.93, 40.02, 40.05, 40.42, 41.23, 41.86, 43.45, 43.53, 47.56, 49.46, 51.92, 114.33, 114.76, 137.60, 138.04, 171.64, 171.76, 173.32, 173.37, 212.78, 212.85. High resolution MS (ESI, m/z): calcd for C₁₅H₂₄NO₄ (M+H)⁺: 282.1700, found: 282.1705.

### Example 7: Non-Enzymatic Reduction of Compound XVIIa into Compound XVIIIa (Main Compound Obtained)

### a) With Sodium Borohydride:

13.76 (48.9 mmol) of compound XVIIa was dissolved in a 150 ml of methanol at −10° C. 1.83 g (48.9 mmol) of sodium borohydride was added and the reaction mixture was stirred until complete reduction of the compound XVIIa (ca 2 hours, HLPC analysis shows a XVIIIa/I ratio of 85/15). The reaction mixture was concentrated under vacuum and the residue was redissolved in 70 ml of ethyl acetate. The solution was washed twice with 100 ml of 1M HCl, 100 ml of saturated sodium bicarbonate and 100 ml of brine before being concentrated under vacuum. The crude product (11.07 g) was purified by column chromatography (silica gel, eluent: DCM to DCM—methanol 94/6 or ethyl acetate—heptane 3/1 to ethyl acetate) to give a colorless liquid. Yield: 80%.

[α]²⁵_{D}: −46.2 (c=1, MeOH). ¹H NMR (400 MHz, CDCl₃—1/1 mixture of two rotamers) δ ppm 1.33-1.46 (m, 2H), 1.50-1.65 (m, 2H), 1.81-1.93 (m, 2H), 2.03-2.14 (m, 3H), 2.22-2.33 (m, 1H), 2.96 (s, 1.5H), 3.10 (s, 1.5H), 3.23-3.36 (m, 1.5H), 3.37-3.42 (m, 1.5H), 3.52-3.61 (m, 0.5H), 3.61-3.68 (m, 1H), 3.70 (s, 1.5H), 3.71 (s, 1.5H), 4.29-4.36 (m, 1H), 4.92-5.06 (m, 2H), 5.07 (d, J=9.32 Hz, 1H), 5.78 and 5.79 (pair of ddt, J=17.04, 10.28, 6.61, 6.61, 1H). ¹³C NMR (101 MHz, CDCl₃—1/1 mixture of two rotamers) δ ppm 25.56, 25.68, 26.22, 28.03, 33.07, 33.14, 34.19, 35.78, 38.66, 39.03, 41.11, 41.36, 41.38, 42.02, 46.93, 47.05, 48.11, 50.32, 51.87, 73.26, 73.32, 114.55, 114.91, 137.76, 138.18, 175.47, 175.48, 176.63, 176.82. High resolution MS (EI, m/z): calcd for C₁₅H₂₅NO₄+H)⁺: 283.1784, found: 283.1738.

### Example 8: Enzymatic Reduction of Compound XVIIa into Compound I and/or Compound XVIIIa (Table of Screened Conditions Below)

A solution of 20 mg of compound XVIIa in 0.1 ml of MTBE is stirred/shaken overnight at room temperature together with a solution of 15 mg of enzyme, 30 mg of glucose, 1 mg of cofactor and 2 mg of GDH in 1.3 ml of 0.1M phosphate buffer pH 7.0. The reaction mixture is then acidified to pH 2 and extracted with 0.3 ml of ethyl acetate. The organic layer is concentrated under vacuum and the residue is analyzed by HPLC.

| example | | | | | |
|---|---|---|---|---|---|
| # | enzyme | cofactor | % XVIIa | % I | % XVIIIa |
| 8-1 | CRED Almac A201 | NADP | 57 | 0 | 43 |
| 8-2 | CRED Almac A301 | NADP | 0 | 76 | 24 |
| 8-3 | CRED Almac A401 | NADP | 10 | 7 | 83 |
| 8-4 | CRED Almac A601 | NADP | 65 | 0 | 35 |
| 8-5 | CRED Almac A701 | NADP | 79 | 0 | 21 |
| 8-6 | CRED Almac A801 | NADP | 0 | 77 | 23 |
| 8-7 | CRED Almac A901 | NADP | 0 | 83 | 17 |
| 8-8 | CRED Almac A121 | NADP | 0 | 76 | 24 |
| 8-9 | CRED Almac A141 | NADP | 10 | 68 | 23 |
| 8-10 | CRED Almac A151 | NAD | 81 | 15 | 4 |
| 8-11 | CRED Almac A161 | NAD | 47 | 41 | 12 |
| 8-12 | CRED Almac A181 | NADP | 1 | 97 | 2 |
| 8-13 | CRED Almac A211 | NADP | 68 | 7 | 25 |
| 8-14 | CRED Almac A231 | NADP | 73.7 | 1 | 25 |
| 8-15 | CRED Almac A241 | NADP | 88 | 1 | 11 |
| 8-16 | CRED Almac A261 | NADP | 10 | 9 | 81 |
| 8-17 | CRED Almac A271 | NADP | 64 | 5 | 31 |
| 8-18 | CRED Almac A281 | NADP | 5 | 39 | 56 |
| 8-19 | CRED Almac A291 | NADP | 73 | 5 | 22 |
| 8-20 | CRED Almac A311 | NADP | 77 | 0 | 23 |
| 8-21 | CRED Almac A341 | NADP | 0 | 71 | 29 |
| 8-22 | CRED Almac A351 | NADP | 0 | 78 | 22 |
| 8-23 | CRED Almac A361 | NADP | 62 | 2 | 36 |
| 8-24 | CRED Almac A371 | NADP | 0 | 35 | 65 |
| 8-25 | CRED Almac A381 | NADP | 77 | 0 | 23 |
| 8-26 | CRED Almac A391 | NADP | 84 | 0 | 16 |
| 8-27 | CRED Almac AN501 | NADP | 7 | 8 | 85 |
| 8-28 | CRED Almac AN701 | NADP | 78 | 1 | 21 |
| 8-29 | CRED Almac AN121 | NADP | 30 | 2 | 68 |
| 8-30 | CRED Almac AN131 | NADP | 26 | 15 | 59 |
| 8-31 | CRED Almac AN151 | NADP | 10 | 23 | 67 |

### Example 9: Reduction of Compound XVIIa into Compound I and/or Compound XVIIIa with Silanes (Table of Screened Conditions Below)

The catalyst and ligand were dissolved or suspended in 2 ml of toluene then 145 mg (0.5 mmol) of compound XVIIa and the silane were added to the mixture and the reaction mixture was stirred at the specified temperature before analysis by either NMR or HPLC.

| example | | % | % | % |
|---|---|---|---|---|
| # | conditions | XVIIa | I | XVIIIa |
| 9-1 | XVIIa, 1.2 equiv, 5 mol % [CuF(PPh₃)₃]•2MeOH, toluene, | 57 | 12 | 31 |
| | rt | | | |
| 9-2 | XVIIa, 1.2 equiv Me₂(EtO)SiH, 5 mol % | 25 | 57 | 18 |
| | [CuF(PPh₃)₃]•2MeOH - dppb, toluene, rt | | | |
| 9-3 | XVIIa, 1.2 equiv (Me₂HSi)₂O, 5 mol % | 9 | 70 | 21 |
| | [CuF(PPh₃)₃]•2MeOH - dppb, toluene, rt | | | |
| 9-4 | XVIIa, 1.2 equiv Me(EtO)₂SiH, 5 mol % | 51 | 35 | 14 |
| | [CuF(PPh₃)₃]•2MeOH - dppb, toluene, rt | | | |
| 9-5 | XVIIa, 1.2 equiv PhSiH₃, 5 mol % [CuF(PPh₃)₃]•2MeOH - | 1 | 75 | 24 |
| | dppb, toluene, rt | | | |
| 9-6 | XVIIa, 1.2 equiv PhSiH₃, 5 mol % [CuF(PPh₃)₃]•2MeOH - | 2 | 47 | 51 |
| | dppm, toluene, rt | | | |
| 9-7 | XVIIa, 1.2 equiv PhSiH₃, 5 mol % [CuF(PPh₃)₃]•2MeOH - | 5 | 60 | 35 |
| | dppe, toluene, rt | | | |
| 9-8 | XVIIa, 1.2 equiv PhSiH₃, 5 mol % [CuF(PPh₃)₃]•2MeOH - | 0 | 47 | 53 |
| | tdppp, toluene, rt | | | |
| 9-9 | XVIIa, 1.2 equiv PhSiH₃, 5 mol % [CuF(PPh₃)₃]•2MeOH - | 4 | 61 | 35 |
| | dppb, toluene, rt | | | |
| 9-10 | XVIIa, 1.2 equiv PhSiH₃, 5 mol % [CuF(PPh₃)₃]•2MeOH - | 3 | 40 | 57 |
| | O-Phdpp, toluene, rt | | | |
| 9-11 | XVIIa, 1.2 equiv PhSiH₃, 5 mol % [CuF(PPh₃)₃]•2MeOH - | 0 | 52 | 48 |
| | O-dppx, toluene, rt | | | |
| 9-12 | XVIIa, 1.2 equiv PhSiH₃, 5 mol % [CuF(PPh₃)₃]•2MeOH - | 0 | 63 | 37 |
| | dppBz, toluene, rt | | | |
| 9-13 | XVIIa, 1.2 equiv PhSiH₃, 5 mol % [CuF(PPh₃)₃]•2MeOH - | 3 | 45 | 52 |
| | dppf, toluene, rt | | | |
| 9-14 | XVIIa, 1.2 equiv PhSiH₃, 5 mol % CuF₂ - dppm, toluene, rt | 7 | 46 | 47 |
| 9-15 | XVIIa, 1.2 equiv PhSiH₃, 5 mol % CuF₂ - dppe, toluene, rt | 20 | 60 | 20 |
| 9-16 | XVIIa, 1.2 equiv PhSiH₃, 5 mol % CuF₂ - tdppp, toluene, rt | 8 | 73 | 19 |
| 9-17 | XVIIa, 1.2 equiv PhSiH₃, 5 mol % CuF₂ - dppb, toluene, rt | 8 | 75 | 17 |
| 9-18 | XVIIa, 1.2 equiv PhSiH₃, 5 mol % CuF₂ - O-Phdpp, | 9 | 20 | 71 |
| | toluene, rt | | | |
| 9-19 | XVIIa, 1.2 equiv PhSiH₃, 5 mol % CuF₂ - O-dppx, toluene, | 7 | 28 | 65 |
| | rt | | | |
| 9-20 | XVIIa, 1.2 equiv PhSiH₃, 5 mol % CuF₂ - dppBz, toluene, | 18 | 38 | 44 |
| | rt | | | |
| 9-21 | XVIIa, 1.2 equiv PhSiH₃, 5 mol % CuF₂ - dppf, toluene, rt | 40 | 13 | 47 |
| 9-22 | XVIIa, 1.2 equiv TMDS, 5 mol % [CuF(PPh₃)₃]•2MeOH - | 0 | 45 | 55 |
| | dppb, toluene, rt | | | |
| 9-23 | XVIIa, 1.2 equiv TMDS, 5 mol % [CuF(PPh₃)₃]•2MeOH - | 0 | 36 | 64 |
| | dppb, toluene, 0° C. | | | |
| 9-24 | XVIIa, 1.2 equiv TMDS, 5 mol % [CuF(PPh₃)₃]•2MeOH - | 0 | 26 | 74 |
| | dppb, toluene, −20° C. | | | |
| 9-25 | XVIIa, 1.2 equiv PhSiH₃, 5 mol % CuF₂ - (S)-BINAP, | 20 | 19 | 61 |
| | toluene, rt | | | |
| 9-26 | XVIIa, 1.2 equiv PhSiH₃, 5 mol % CuF₂ - (R)-BINAP, | 11 | 21 | 68 |
| | toluene, rt | | | |
| 9-27 | XVIIa, 1.2 equiv PhSiH₃, 5 mol % CuF₂ - (S)-3,5- | 5 | 31 | 64 |
| | tBuMeOBIPHEP, toluene, rt | | | |
| 9-28 | XVIIa, 1.2 equiv PhSiH₃, 5 mol % CuF₂ - (R)-3,5- | 39 | 20 | 41 |
| | tBuMeOBIPHEP, toluene, rt | | | |
| 9-29 | XVIIa, 1.2 equiv PhSiH₃, 5 mol % CuF₂ - (S)- | 15 | 20 | 65 |
| | MeOBIPHEP, toluene, rt | | | |
| 9-30 | XVIIa, 1.2 equiv PhSiH₃, 5 mol % CuF₂ - (S,S)-DIOP, | 12 | 73 | 15 |
| | toluene, rt | | | |
| 9-31 | XVIIa, 1.2 equiv PhSiH₃, 5 mol % CuF₂ - (R,R)-norphos, | 35 | 28 | 37 |
| | toluene, rt | | | |
| 9-32 | XVIIa, 1.2 equiv PhSiH₃, 5 mol % CuF₂ - (S,S)-DIPAMP, | 9 | 48 | 43 |
| | toluene, rt | | | |
| 9-33 | XVIIa, 1.2 equiv PhSiH₃, 5 mol % CuF₂ - (S,S)-chiraphos, | 5 | 45 | 50 |
| | toluene, rt | | | |
| 9-34 | XVIIa, 1.2 equiv PhSiH₃, 5 mol % [CuF(PPh₃)₃]•2MeOH - | 64 | 21 | 15 |
| | (R,R)-norphos, toluene, rt | | | |
| 9-35 | XVIIa, 1.2 equiv PhSiH₃, 5 mol % [CuF(PPh₃)₃]•2MeOH - | 0 | 44 | 56 |
| | (S,S)-DIPAMP, toluene, rt | | | |
| 9-36 | XVIIa, 1.2 equiv PhSiH₃, 5 mol % [CuF(PPh₃)₃]•2MeOH - | 0 | 48 | 52 |
| | (S,S)-chiraphos, toluene, rt | | | |
| 9-37 | XVIIa, 1.2 equiv TMDS, 5 mol % [CuF(PPh₃)₃]•2MeOH - | 69 | 16 | 15 |
| | (R,R)-norphos, toluene, rt | | | |
| 9-38 | XVIIa, 1.2 equiv TMDS, 5 mol % [CuF(PPh₃)₃]•2MeOH - | 0 | 64 | 36 |
| | (S,S)-DIPAMP, toluene, rt | | | |
| 9-39 | XVIIa, 1.2 equiv TMDS, 5 mol % [CuF(PPh₃)₃]•2MeOH - | 0 | 48 | 52 |
| | (S,S)-chiraphos, toluene, rt | | | |
| 9-40 | XVIIa, 1.2 equiv PhSiH₃, 5 mol % CuF₂ - (S,S)-DIOP, | 3 | 87 | 10 |
| | toluene, 0° C. | | | |
| 9-41 | XVIIa, 1.2 equiv PhSiH₃, 5 mol % CuF₂ - dppb, MTBE, rt | 7 | 71 | 22 |
| 9-42 | XVIIa, 1.2 equiv PhSiH₃, 5 mol % CuF₂ - dppb, n-hexane, | 7 | 64 | 29 |
| | rt | | | |
| 9-43 | XVIIa, 1.2 equiv PhSiH₃, 5 mol % CuF₂ - dppb, THF, rt | 7 | 64 | 29 |
| 9-44 | XVIIa, 1.2 equiv PhSiH₃, 5 mol % CuF₂ - dppb, Et₂O, rt | 50 | 40 | 10 |
| 9-45 | XVIIa, 1.2 equiv PhSiH₃, 5 mol % CuF₂ - dppb, | 53 | 36 | 11 |
| | chlorobenzene, rt | | | |
| 9-46 | XVIIa, 1.2 equiv PhSiH₃, 5 mol % CuF₂ - dppb, | 23 | 58 | 19 |
| | fluorobenzene, rt | | | |
| 9-47 | XVIIa, 1.2 equiv PhSiH₃, 5 mol % [CuF(PPh₃)₃]•2MeOH - | 0 | 73 | 27 |
| | dppb, MTBE, rt | | | |
| 9-48 | XVIIa, 1.2 equiv PhSiH₃, 5 mol % [CuF(PPh₃)₃]•2MeOH - | 13 | 48 | 39 |
| | dppb, n-hexane, rt | | | |
| 9-49 | XVIIa, 1.2 equiv PhSiH₃, 5 mol % [CuF(PPh₃)₃]•2MeOH - | 20 | 56 | 24 |
| | dppb, THF, rt | | | |
| 9-50 | XVIIa, 1.2 equiv PhSiH₃, 5 mol % [CuF(PPh₃)₃]•2MeOH - | 0 | 77 | 23 |
| | dppb, Et₂O, rt | | | |
| 9-51 | XVIIa, 1.2 equiv PhSiH₃, 5 mol % [CuF(PPh₃)₃]•2MeOH - | 0 | 67 | 33 |
| | dppb, chlorobenzene, rt | | | |
| 9-52 | XVIIa, 1.2 equiv PhSiH₃, 5 mol % [CuF(PPh₃)₃]•2MeOH - | 0 | 69 | 31 |
| | dppb, fluorobenzene, rt | | | |
| 9-53 | XVIIa, 1.2 equiv TMDS, 5 mol % [CuF(PPh₃)₃]•2MeOH - | 0 | 69 | 31 |
| | dppb, MTBE, rt | | | |
| 9-54 | XVIIa, 1.2 equiv TMDS, 5 mol % [CuF(PPh₃)₃]•2MeOH - | 0 | 45 | 55 |
| | dppb, n-hexane, rt | | | |
| 9-55 | XVIIa, 1.2 equiv TMDS, 5 mol % [CuF(PPh₃)₃]•2MeOH - | 60 | 24 | 16 |
| | dppb, THF, rt | | | |
| 9-56 | XVIIa, 1.2 equiv TMDS, 5 mol % [CuF(PPh₃)₃]•2MeOH - | 11 | 64 | 25 |
| | dppb, Et₂O, rt | | | |
| 9-57 | XVIIa, 1.2 equiv TMDS, 5 mol % [CuF(PPh₃)₃]•2MeOH - | 66 | 22 | 12 |
| | dppb, chlorobenzene, rt | | | |
| 9-58 | XVIIa, 1.2 equiv TMDS, 5 mol % [CuF(PPh₃)₃]•2MeOH - | 53 | 32 | 15 |
| | dppb, fluorobenzene, rt | | | |
| 9-59 | XVIIa, 1.2 equiv PhSiH₃, 5 mol % CuF₂ - DCyPh, toluene, | 43 | 10 | 47 |
| | rt | | | |
| 9-60 | XVIIa, 1.2 equiv PhSiH₃, 5 mol % CuF₂ - d(t-Bu)pf, | 20 | 26 | 54 |
| | toluene, rt | | | |
| 9-61 | XVIIa, 1.2 equiv PhSiH₃, 5 mol % [CuF(PPh₃)₃]•2MeOH - | 6 | 17 | 77 |
| | DCyPh, toluene, rt | | | |
| 9-62 | XVIIa, 1.2 equiv PhSiH₃, 5 mol % [CuF(PPh₃)₃]•2MeOH - | 45 | 18 | 37 |
| | d(t-Bu)pf, toluene, rt | | | |
| 9-63 | XVIIa, 1.2 equiv PhSiH₃, 5 mol % CuF₂ - (R)-BINAP, | 13 | 21 | 66 |
| | toluene, rt | | | |
| 9-64 | XVIIa, 1.2 equiv PhSiH₃, 5 mol % CuF₂ - (S)-BINAP, | 56 | 10 | 34 |
| | toluene, rt | | | |
| 9-65 | XVIIa, 1.2 equiv PhSiH₃, 5 mol % CuF₂ - (R)-3,5- | 7 | 38 | 55 |
| | tBuMeOBIPHEP, toluene, rt | | | |
| 9-66 | XVIIa, 1.2 equiv PhSiH₃, 5 mol % CuF₂ - (S)-3,5- | 3 | 25 | 72 |
| | tBuMeOBIPHEP, toluene, rt | | | |
| 9-67 | XVIIa, 1.2 equiv PhSiH₃, 5 mol % CuF₂ - (S,S)-DIOP, | 3 | 82 | 15 |
| | toluene, rt | | | |
| 9-68 | XVIIa, 1.2 equiv PhSiH₃, 5 mol % CuF₂ - (S,S)-DIPAMP, | 43 | 17 | 40 |
| | toluene, rt | | | |
| 9-69 | XVIIa, 1.2 equiv PhSiH₃, 5 mol % [CuF(PPh₃)₃]•2MeOH - | 78 | 6 | 16 |
| | (R)-BINAP, toluene, rt | | | |
| 9-70 | XVIIa, 1.2 equiv PhSiH₃, 5 mol % [CuF(PPh₃)₃]•2MeOH - | 57 | 13 | 30 |
| | (S)-BINAP, toluene, rt | | | |
| 9-71 | XVIIa, 1.2 equiv PhSiH₃, 5 mol % [CuF(PPh₃)₃]•2MeOH - | 2 | 39 | 59 |
| | (R)-3,5-tBuMeOBIPHEP, toluene, rt | | | |
| 9-72 | XVIIa, 1.2 equiv PhSiH₃, 5 mol % [CuF(PPh₃)₃]•2MeOH - | 2 | 24 | 74 |
| | (S)-3,5-tBuMeOBIPHEP, toluene, rt | | | |
| 9-73 | XVIIa, 1.2 equiv PhSiH₃, 5 mol % [CuF(PPh₃)₃]•2MeOH - | 0 | 74 | 26 |
| | (S,S)-DIOP, toluene, rt | | | |
| 9-74 | XVIIa, 1.2 equiv PhSiH₃, 5 mol % [CuF(PPh₃)₃]•2MeOH - | 3 | 43 | 54 |
| | (S,S)-DIPAMP, toluene, rt | | | |
| 9-75 | XVIIa, 2 equiv PhSiH₃, 3 mol % IMES-Cu-DBM, toluene, | 7 | 16 | 77 |
| | 80° C. | | | |
| 9-76 | XVIIa, 2 equiv PhSiH₃, 3 mol % SIMES-Cu-DBM, | 7 | 6 | 87 |
| | toluene, 80° C. | | | |
| 9-77 | XVIIa, 2 equiv PhSiH₃, 3 mol % IAD-Cu-DBM, toluene, | 4 | 5 | 91 |
| | 80° C. | | | |
| 9-78 | XVIIa, 2 equiv PhSiH₃, 3 mol % IMES-Cu-DBM, toluene, | 0 | 20 | 80 |
| | 80° C. | | | |
| 9-79 | XVIIa, 2 equiv PhSiH₃, 3 mol % SIMES-Cu-DBM, | 3 | 15 | 82 |
| | toluene, 80° C. | | | |

### Structure of Ligands Used:

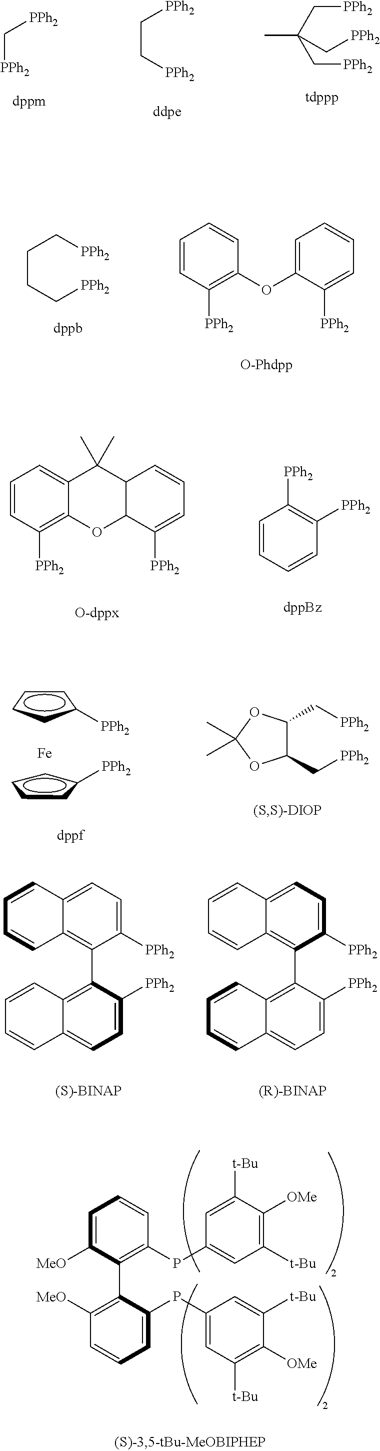

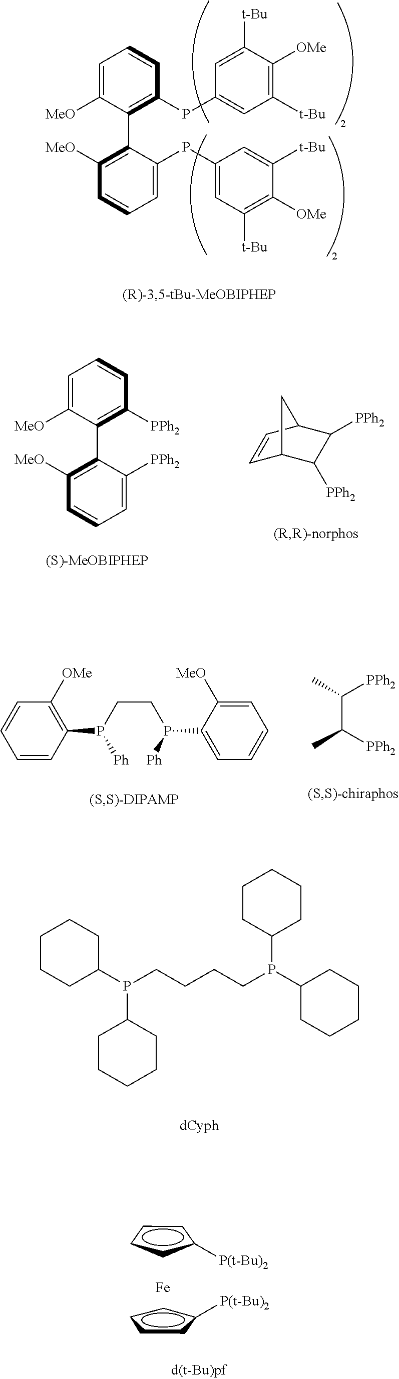

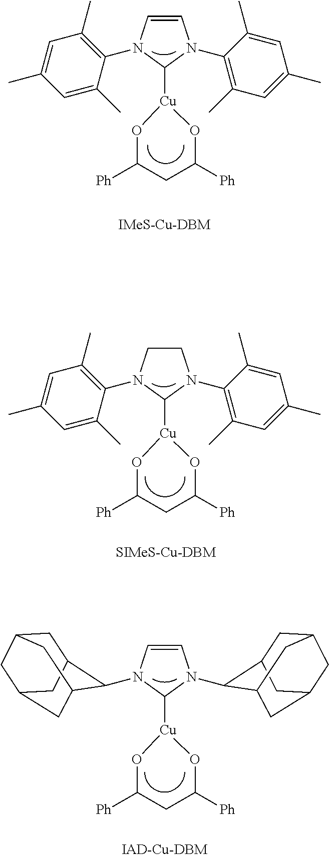

### Example 10: Reduction of Compound XVIIa into Compound I and/or Compound XVIIIa with Either Formic Acid or Isopropanol (Table of Screened Conditions Below)

The metal complex and ligand were dissolved or suspended in 2 ml of IPA with potassium hydroxide then 145 mg (0.5 mmol) of compound XVIIa was added and the reaction mixture was stirred at the specified temperature before analysis by either NMR or HPLC.

| Ex. | | % | % | % |
|---|---|---|---|---|
| # | conditions | XVIIa | I | XVIIIa |
| 10-1 | XVIIa, 5 mol %[RuCl₂(p-cym)]₂ - | 4 | 66 | 30 |
| | 1R,2S-AA, 5 mol % KOH, IPA, rt | | | |
| 10-2 | XVIIa, 5 mol %[RuCl₂(p-cym)]₂ - | 0 | 69 | 31 |
| | 1S,2R-AA, 5 mol % KOH, IPA, rt | | | |
| 10-3 | XVIIa, 5 mol %[RuCl₂(p-cym)]₂ - | 0 | 40 | 60 |
| | NV107, 5 mol % KOH, IPA, rt | | | |
| 10-4 | XVIIa, 5 mol %[RuCl₂(p-cym)]₂ - A6, | 0 | 70 | 30 |
| | 5 mol % KOH, IPA, rt | | | |
| 10-5 | XVIIa, 5 mol %[RuCl₂(p-cym)]₂ - A9, | 0 | 69 | 31 |
| | 5 mol % KOH, IPA, rt | | | |
| 10-6 | XVIIa, 5 mol %[RuCl₂(p-cym)]₂ - A11, | 0 | 71 | 29 |
| | 5 mol % KOH, IPA, rt | | | |
| 10-7 | XVIIa, 5 mol %[RuCl₂(p-cym)]₂ - A3, | 0 | 62 | 38 |
| | 5 mol % KOH, IPA, rt | | | |
| 10-8 | XVIIa, 5 mol %[RuCl₂(p-cym)]₂ - | 11 | 41 | 48 |
| | Ts-DPN, 5 mol % KOH, IPA, rt | | | |

### Ligands Used:

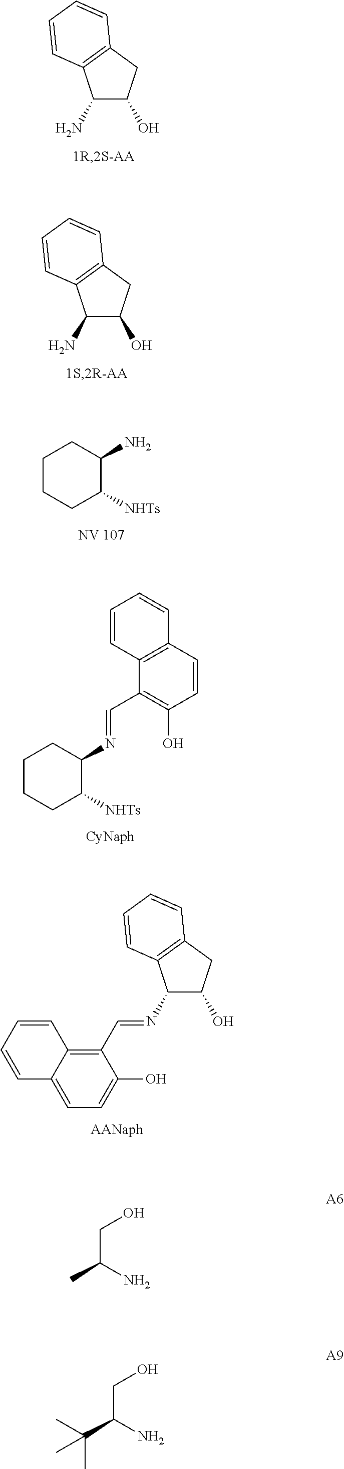

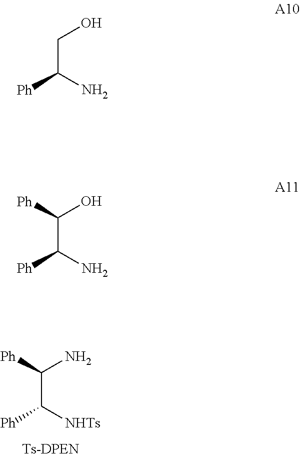

### Example 11: Preparation of Compound I from Compound IV without Isolation (which May be Advantageous)

a) Esterification of Compound IV into Compound XVa

0.83 μl (13.7 mol) of concentrated sulfuric acid is added to a solution of 4.5 kg (26.1 mol) of compound IV in 20 μl of methanol and 30 μl of toluene slowly to maintain the temperature below 30° C. The reaction mixture is then stirred at 70° C. until complete conversion of the compound IV (ca 1-3 h) before concentration to half of its original volume (24 μl distilled). After cooling to 30° C., 25 μl of water and 61 of MeTHF are added and the reaction mixture is stirred an hour before phase decantation. The water layer is discarded and the organic one is washed with 10 μl of water, 10 μl of saturated sodium bicarbonate solution and 5 μl of brine then concentrated to a final volume of about 9 μl. Assay of the so-obtained solution indicates 4.5 kg of compound XVa was obtained. This solution is used as such in the next reaction. Yield: 86%.

### b) Kinetic Resolution of Compound XVa Towards Compound (R,R)-XVIa

A solution of 4.5 kg (24.1 mol) of compound XVa in MeTHF—toluene is added to a well stirred 0.1 M pH 6.5 phosphate buffer solution (38.2 μl). 450 g of 10 w/w % CAL-B lysate is added and the reaction mixture is stirred for 24 h with a constant pH adjustment between 6.4 and 6.6 by regular addition of 4M sodium hydroxide solution (2.8 μl in total). 875 g of celite and 22.5 μl of MeTHF are added and the reaction mixture is filtered through a pad of celite. The pH of the biphasic filtrate is adjusted between 6.5 and 7 with either 4 M sodium hydroxide or 5M hydrochloric acid solutions, the layers are separated and the aqueous one is washed with 4 times 22.5 μl of MeTHF (extraction of the compound (S,S)-XVa) before addition of 5M hydrochloric acid solution to reach pH 2. The acidified water layer is extracted twice with 22.5 μl of MeTHF (extraction of the compound (R,R)-XVIa). The organic layer is concentrated to a final volume of about 9 μl. Assay of the so-obtained solution indicates 1.55 kg of compound (R,R)-XVIa was obtained with >98% chemical purity and 94% ee. Yield: 37%. The solution of compound (R,R)-XVIa is used as such in the next step.

c) Amide Coupling from Compound (R,R)-XVIa to Compound XVIIa

A solution of 1.55 kg (8.33 mol) of compound (R,R)-XVIa in MeTHF is added to a stirred suspension of 1.48 kg (9.16 mol) of CDI in 15.5 μl of MeTHF over 15 min. After conversion of the compound (R,R)-XVIa into its activated form, the reaction mixture is cooled to 10° C. and 1.22 kg (10.83 mol) of compound VIII is slowly added to keep the temperature between 10 and 15° C. The reaction mixture is then warmed to 20° C. and stirred complete conversion (ca 1 h) before being cooled to 10° C. and washed twice with 7.7 μl of 1 M hydrochloric acid solution, twice with 7.7 μl of saturated sodium bicarbonate solution and 7.7 μl of brine. The organic layer is concentrated and the residue is redissolved with 3 μl of toluene. Assay of the solution indicates 2 kg of compound XVIIa was obtained. This solution is used as such in the next step. Yield: 85%.

d) Enzymatic Reduction of Compound XVIIa into Compound I

A solution of 1.2 kg (4.3 mol) of compound XVIIa in toluene is added to a solution of 1.15 kg (6.33 mol) of D-glucose in 36 μl of 0.1 M phosphate buffer pH 7.0 solution. After a few minutes of stirring, 0.36 kg (0.49 mol) of NADP, 0.12 kg of GDH and 0.3 kg of CRED Almac A181 are added. The reaction mixture is stirred 48 h at 20° C. with constant pH adjustment by regular addition of 4 M sodium hydroxide solution (1.07 μl in total). 24 l of toluene, 2 kg of celite and 5 kg of sodium chloride are added and the reaction mixture is stirred 15 minutes then filtered through a pad of celite. The two layers of the filtrate are separated and the water layer is extracted with 24 μl of toluene. The combined two organic layers are washed with 24 μl of brine, filtered through a pad of celite and concentrated to obtain 14.6 kg of a 25 w % solution of compound I in toluene with a 97.3% chemical purity and >98.5% diastereoisomeric excess. The so-obtained compound I in toluene can be converted into the compound II following described procedures (eg example 6b in WO2010072742(A1)). Yield: 85%.

### Example 12: Enzymatic Diastereoselective Reduction of Compound (R,R)-XVIa into Either Compound XIXa or Compound XXa (Table of Screened Conditions Below)

A solution of 20 mg of compound (R,R)-XVIa in 0.1 ml of MTBE was stirred/shaken overnight at room temperature together with a solution of 15 mg of enzyme, 30 mg of glucose, 1 mg of cofactor and 2 mg of GDH in 1.3 ml of 0.1M phosphate buffer pH 7.0. the reaction mixture was then acidified to pH 2 and extracted with 0.3 ml of ethyl acetate. The organic layer was concentrated under vacuum and the residue was analyzed by HPLC to assess the yield and the stereoselectivity.

| | | | LC | | | | |
|---|---|---|---|---|---|---|---|
| | | | % | | | | % d.e. |
| example | | co- | (R,R)- | % | % | % | (main |
| # | enzyme | factor | XVIa | XIXa | XXa | yield | isomer) |
| 12-1 | CRED | NADP | 0 | 4 | 96 | 100 | 93 |
| | Almac A301 | | | | | | (Xxa) |
| 12-2 | CRED | NADP | 20 | 0 | 80 | 80 | 100 |
| | Almac A501 | | | | | | (Xxa) |
| 12-3 | CRED | NADP | 0 | 0 | 100 | 100 | 100 |
| | Almac A601 | | | | | | (Xxa) |
| 12-4 | CRED | NADP | 0 | 14 | 86 | 100 | 71 |
| | Almac A801 | | | | | | (Xxa) |
| 12-5 | CRED | NADP | 0 | 18 | 83 | 100 | 65 |
| | Almac A901 | | | | | | (Xxa) |
| 12-7 | CRED | NADP | 21 | 17 | 62 | 79 | 58 |
| | Almac A121 | | | | | | (Xxa) |
| 12-8 | CRED | NADP | 0 | 0 | 100 | 100 | 100 |
| | Almac A141 | | | | | | (Xxa) |
| 12-9 | CRED | NAD | 79 | 2 | 20 | 22 | 86 |
| | Almac A151 | | | | | | (Xxa) |
| 12-10 | CRED | NAD | 44 | 11 | 46 | 56 | 62 |
| | Almac A161 | | | | | | (Xxa) |
| 12-11 | CRED | NADP | 48 | 0 | 52 | 52 | 100 |
| | Almac A261 | | | | | | (Xxa) |
| 12-12 | CRED | NADP | 71 | 14 | 15 | 29 | 3 |
| | Almac A281 | | | | | | (Xxa) |
| 12-13 | CRED | NADP | 0 | 0 | 100 | 100 | 100 |
| | Almac A341 | | | | | | (Xxa) |
| 12-14 | CRED | NADP | 0 | 14 | 86 | 100 | 73 |
| | Almac A351 | | | | | | (Xxa) |
| 12-15 | CRED | NADP | 0 | 28 | 72 | 100 | 44 |
| | Almac A361 | | | | | | (Xxa) |

### Example 13: Formation of Compound XVIIIa from Compound (R,R)-XVIa (Preparative Scale)

a) Enzymatic Diastereoselective Reduction of Compound (R,R)-XVIa into Compound XXa:

500 mg of compound (R,R)-XVIa was reduced under the conditions used for the screening of enzymes using the CRED Almac A301 and gave 500 mg of compound XXa (XXa/XIXa ratio: 97/3) which was used as such in the next step. Yield: quantitative. High resolution MS (ESI, m/z): calcd for C₈H₁₂NaO₅ (M+Na)⁺: 211.0577, found: 211.0565.

### b) Amide Coupling (Compound XXa+Compound VIII→Compound XVIIIa):

Compound XXa was converted into compound XVIIIa with compound VIII and EEDQ following the procedure described in the example 6a. Yield: quantitative, XVIIIa/I ratio: 96/4.

### Example 14: Reduction of Compound (R,R)-XVa into Compound (R,R)-XXIa

### a) With Sodium Borohydride:

Compound (R,R)-XVa was dissolved in methanol and reduced with sodium borohydride to give compound (R,R)-XXIa in 89% yield after standard aqueous workup and chromatographic purification.

### b) With Hydrogen and Rh/Alumina:

A solution of 25 g (125 mmol) of compound (R,R)-XVa was stirred overnight under hydrogen atmosphere in the presence of 6.43 g of 5 w % Rh/alumina catalyst. After filtration of the catalyst, the solution was concentrated under vacuum to give 21.62 g of crude compound (R,R)-XXIa as an oil. Yield: 86%.

### Example 15: Selective Hydrolysis of Compound (R,R)-XXIa into Either Compound XIXa or Compound XXa (Table of Screened Conditions Below)

A solution of 20 mg of compound (R,R)-XXIa in 0.1 ml of MTBE is stirred/shaken overnight at room temperature together with a solution of 10 mg of enzyme in 1 ml of 0.1M phosphate buffer pH 7.0. The reaction mixture is then acidified to pH 1 with concentrated HCl and the compounds of interest (compounds (R,R)-XXIa, XIXa and XXa) are extracted with 0.3 ml of ethyl acetate. The organic solution is diluted and analyzed by HPLC.

| exam- | | % (R,R)- | % | % |
|---|---|---|---|---|
| ple # | Enzyme | XXIa | XIXa | XXa |
| 15-1 | lipase C2 from Candida Cylindrica | 57 | 17 | 26 |
| 15-2 | lipase from Mucor Javanicus | 88 | 7 | 6 |
| 15-3 | protease from Aspergillus Oryzae | 85 | 9 | 5 |
| 15-4 | protease from Aspergillus Melleus | 68 | 30 | 2 |
| 15-5 | protease from Bacillus Subtilis | 88 | 10 | 1 |
| 15-6 | aminoacylase from Aspergillus sp. | 89 | 9 | 2 |
| 15-7 | lipase B from Candida Antartica | 59 | 25 | 15 |
| | (CAL-B) | | | |
| 15-8 | lipase A from Candida Antartica | 94 | 2 | 4 |
| | (CAL-A) | | | |
| 15-9 | ficin from Fiscus sp. | 52 | 26 | 22 |
| 15-10 | papain from Papaya Carica | 92 | 5 | 2 |
| 15-11 | immobilized lipase B from Candida | 85 | 10 | 5 |
| | Antartica (immobilized CAL-B) | | | |
| 15-12 | protease from Bacillus Lentus | 60 | 36 | 4 |
| 15-13 | immobilized protease from Bacillus | 10 | 86 | 4 |
| | Lentus | | | |
| 15-14 | protease from Bacillus Licheniformis | 50 | 34 | 16 |
| 15-15 | immobilized protease from | 65 | 33 | 2 |
| | Bacillus Licheniformis | | | |
| 15-16 | esterase from pig liver (PLE) | 55 | 14 | 31 |

### Example 16: Selective Hydrolysis of Compound (R,R)-XXIa into Compound XIXa

500 mg of compound (R,R)-XXIa was hydrolyzed with immobilized protease from Bacillus Lentus under the conditions used for the screening and deliver 200 mg of compound XIXa (40% yield, XIXa/XXa ratio: 93/7).

### Example 17: Hydrolysis of Compound XVIIIa into Compound XXIII

A solution of 1.21 g (28.8 mmol) of lithium hydroxide monohydrate in 14 ml of water was added to a solution of 8.00 g (28.2 mmol) of compound XVIIIa in 28 ml of THF. The biphasic mixture was stirred thoroughly for about two hours at room temperature before 28 ml of isopropyl acetate was added. After decantation, the organic layer is discarded and the water layer is neutralized with 2.6 ml of concentrated hydrochloric acid. The compound XXIII is extracted from the acidic water layer with 28 ml of isopropyl acetate. The organic layer is dried over magnesium sulfate, filtered and concentrated under vacuum to give 6.5 g of crude compound XIII as an oil. Yield: 85%.

[α]²⁵_{D}: −46.4 (c=1, MeOH). ¹H NMR (400 MHz, CDCl₃) δ ppm 1.32-1.47 (m, 2H) 1.49-1.67 (m, 2H) 1.85-2.00 (m, 2H) 2.04-2.16 (m, 3H) 2.25-2.38 (m, 1H) 2.96 (s, 1.5H) 3.12 (s, 1.5H) 3.20-3.48 (m, 2.5H) 3.56-3.73 (m, 1.5H) 4.39 (t, J=4.03 Hz, 1H) 4.91-5.06 (m, 2H) 5.78 (2×ddt, J=17.06, 10.26, 6.61, 6.61 Hz, 1H) 8.22 (br. s., 1H).

### Example 18: Compound XXIVa

11.1 ml (63.6 mmol) of ethyldiisopropylamine was added dropwise to a solution of 20 g (63.6 mmol) of compound XXII and 6.5 ml (70 mmol) of POCl₃ in 127 mol of acetonitrile. The reaction mixture was then refluxed until complete conversion of the compound XXII (ca 3 h) before being cooled to 45° C. 64 ml of water was added and the resulting suspension was cooled to 35° C. and filtered. The filter cake was wash with 20 ml of acetonitrile and 20 ml of water and dried under vacuum to give 11.23 g of compound XXIVa (yellow solid). Yield: 53%.

Characterization data (CAS 1193272-59-1) fits with the literature (eg: US 20090269305). High resolution MS (ESI, m/z): calcd for C₁₇H₁₈ClN₂OS (M+H)⁺: 333.0823, found: 333.0801.

### Example 19: Compound XXIVb

20 g (63.6 mmol) of compound XXII was added by portions to a solution of 20.06 g (70 mmol) of POBr₃ in 127 mol of acetonitrile. After complete addition of the compound XXII, 11.1 ml (63.6 mmol) of ethyldiisopropylamine was added dropwise and the reaction mixture was refluxed until complete conversion (ca 1 h) before being cooled to 45° C. 64 ml of water was added and the resulting suspension was cooled to 25° C. and filtered. The filter cake was wash with 20 ml of acetonitrile and 20 ml of water and dried under vacuum to give 21.9 g of compound XXIVa (yellow solid). Yield: 91%.

High resolution MS (ESI, m/z): calcd for C₁₇H₁₈BrN₂OS (M+H)⁺: 377.0318, found: 377.0337.

### Example 20: Compound XXIVc

a) A solution/suspension of 0.88 g (2.94 mmol) of compound XXIVa and 883 mg (5.89 mmol) of sodium iodide in 6 ml of 1,4-dioxane is stirred overnight at 110° C. then cooled to room temperature before addition of 11 ml of water and 6 ml of dichloromethane. The two layers are separated and the aqueous one is extracted with 6 ml of dichloromethane. The combined organic layers are dried over magnesium sulfate, filtered and concentrated under vacuum to dryness. The crude residue is recrystallized from 4 ml of methylisobutylketone to yield 660 mg of compound XXIc as a white solid. A second fraction of 420 mg of compound XXIc is obtained after evaporation of the mother-liquor and recrystallization of the residue from methylisobutylketone. Combined yield: 86%.

b) A solution/suspension of 40 g (120 mmol) of compound XXIVa and 36 g (240 mmol) of sodium iodide in 240 ml of 1,4-dioxane is dried azeotropically (about 50 ml of solvent is distilled off while the internal temperature raises from 90° C. to 100° C.) before addition of 0.54 ml (6 mmol) of concentrated aqueous HCl. After overnight reflux, the reaction mixture is concentrated (160 ml of solvent distilled off) then cooled to 40° C. before addition of 120 ml of water and 180 ml of dichloromethane and 0.44 ml (8.4 mmol) of 50 w % aqueous sodium hydroxide. The two layers are separated at 40° C. and the organic one is dried over magnesium sulfate, filtered and concentrated under vacuum to dryness to afford 49.9 g of compound XXIVc as a light yellow solid. Yield: 98%.

¹H NMR (400 MHz, CDCl₃) δ ppm 1.38 (s, 3H), 1.39 (s, 3H), 2.66 (s, 3H), 3.18 (hept d, J=6.9, 0.8 Hz, 1H), 3.69 (s, 2H—1/2 dioxane), 3.95 (s, 3H), 7.01 (d, J=1.0 Hz, 1H), 7.25 (d, J=9.3 Hz, 1H), 7.80 (d, J=9.1 Hz, 1H), 8.70 (s, 1H). ¹³C NMR (101 MHz, CDCl₃) δ ppm 9.91, 22.44, 31.09, 56.15, 67.04, 112.46, 114.50, 114.57, 122.47, 125.30, 126.29, 129.99, 146.40, 149.93, 158.24, 165.25, 167.57. High resolution MS (ESI, m/z): calcd for C₁₇H₁₈IN₂OS (M+H)⁺: 425.0179, found: 425.0175.

### Example 21: Compound XXIVd

3.1 ml (38.2 mmol) of pyridine and 2.7 ml (35 mmol) of methanesulfonyl chloride are added to a dried suspension of 10 g (38.1 mmol) of compound XXII in 105 ml of dichloromethane kept at 0° C. The reaction mixture is stirred at 0° C. until complete reaction then washed successively with 16 ml of 1 M hydrochloric acid solution, 32 ml of water, 16 ml of 0.5 M sodium hydroxide solution and water, dried over magnesium sulfate, filtered and concentrated under vacuum. The residue is recrystallized from 31 ml of MIBK to give 9.71 g of compound XXIVd as a white solid. Yield: 78%. Mp 134.5° C. ¹H NMR (400 MHz, CDCl₃) δ ppm 1.37 (s, 3H), 1.38 (s, 3H), 2.69 (s, 3H), 3.17 (hept d, J=6.9, 0.6 Hz, 1H), 3.33 (s, 3H), 3.97 (s, 3H), 7.03 (d, J=0.8 Hz, 1H), 7.32 (d, J=9.3 Hz, 1H), 7.94 (d, J=9.1 Hz, 1H), 8.12 (s, 1H). ¹³C NMR (101 MHz, CDCl₃) δ ppm 9.87, 22.40, 31.06, 38.62, 56.18, 106.31, 114.19, 114.72, 117.18, 119.85, 119.85, 122.64, 149.32, 151.77, 153.51, 158.50, 165.33, 168.04. High resolution MS (ESI, m/z): calcd for C₁₈H₂₁N₂O₄S₂ (M+H)⁺: 393.0937, found: 393.0949.

### Example 22: Compound XXIVe

3.1 ml (38.2 mmol) of pyridine and 6.67 g (35 mmol) of p-toluenesulfonyl chloride are added to a dried suspension of 10 g (38.1 mmol) of compound XXII in 105 ml of dichloromethane kept at 25° C. The reaction mixture is stirred at 25° C. until complete reaction then diluted with 32 ml of dichloromethane, washed successively at 40° C. with 16 ml of 1 M hydrochloric acid solution, 32 ml of water, 16 ml of 0.5 M sodium hydroxide solution and water, dried over magnesium sulfate, filtered and concentrated under vacuum. The residue is recrystallized from 64 ml of MIBK to give 11.51 g of compound XXIVe as a off-white solid. Yield: 77%.

Mp 155.8° C. ¹H NMR (400 MHz, CDCl₃) δ ppm 1.35 (s, 3H), 1.37 (s, 3H), 2.43 (s, 3H), 2.67 (s, 3H), 3.13 (hept d, J=6.9, 0.7 Hz, 1H), 3.97 (s, 3H), 7.01 (d, J=1.0 Hz, 1H), 7.25 (d, J=9.3 Hz, 1H), 7.33 (d, J=8.1 Hz, 2H), 7.80 (s, 1H), 7.83 (d, J=9.3 Hz, 1H), 7.86 (d, J=8.3 Hz, 2H). ¹³C NMR (101 MHz, CDCl₃) δ ppm 9.83, 21.71, 22.33, 31.03, 56.22, 106.62, 113.94, 114.57, 117.52, 120.14, 122.38, 128.62, 130.01, 132.42, 145.85, 149.29, 151.66, 153.94, 158.35, 165.11, 168.06. High resolution MS (ESI, m/z): calcd for C₂₄H₂₅N₂O₄S₂ (M+H)⁺: 469.1250, found: 469.1208.

### Example 23: Compound XXIVf

4.4 ml (38.2 mmol) of 2,6-lutidine and 5.9 ml (35 mmol) of trifluoromethanesulfonic anhydride are added slowly to a dried suspension of 10 g (38.1 mmol) of compound XXII in 105 ml of dichloromethane kept at 0° C. The reaction mixture is stirred at 0° C. until complete reaction then washed successively with 16 ml of 1 M hydrochloric acid solution, 32 ml of water, 16 ml of 0.5 M sodium hydroxide solution and water, dried over magnesium sulfate, filtered and concentrated under vacuum. The residue is recrystallized from 32 ml of MIBK to give 9.90 g of compound XXIVf as a white solid. Yield: 70%.

Mp 134.5° C. ¹H NMR (400 MHz, CDCl₃) δ ppm 1.38 (s, 3H), 1.39 (s, 3H), 2.70 (s, 3H), 3.19 (hept d, J=6.9, 0.9 Hz, 1H), 4.00 (s, 3H), 7.06 (d, J=0.8 Hz, 1H), 7.39 (d, J=9.3 Hz, 1H), 7.88 (d, J=9.3 Hz, 1H), 8.12 (s, 1H). ¹³C NMR (101 MHz, CDCl₃) δ ppm 9.95, 22.40, 31.09, 56.23, 106.34, 118.69 (q, J=320.6 Hz), 114.72, 115.05, 116.17, 119.04, 123.05, 149.43, 151.83, 153.51, 158.84, 165.53, 167.44. High resolution MS (ESI, m/z): calcd for C₁₈H₁₈F₃N₂O₄S₂ (M+H)⁺: 447.0508, found: 447.0508.

### Example 24: Compounds II and XXV from Reaction Between Compound XVIIIa and/or Compound XXIII, and Compounds XXIV (Table of Screened Conditions)

| | | LC (% of |
|---|---|---|
| # | Conditions | product) |
| 24-1 | XVIIIa (100 mg), XXIVb (133 mg), tBuONa (68 mg), DMF (2 × | 15% XXV |
| | 1.4 ml), 60° C. to 100° C., 2 days | |
| 24-2 | XVIIIa (50 mg), XXIVb (67 mg), tBuONa (36 mg), DMF (0.7 | 5% XXV |
| | ml), 110° C., o/n | |
| 24-3 | XVIIIa (50 mg), XXIVb (67 mg), tBuONa (36 mg), 1,4- | 20% XXV |
| | dioxane (0.7 ml), 110° C., o/n | |
| 24-4 | XVIIIa (50 mg), XXIVd (69 mg), tBuONa (68 mg), 1,4- | 8% XXV |
| | dioxane (0.7 ml), 10° C., o/n | |
| 24-5 | XVIIIa (57 mg), XXIVb (67 mg), tBuONa (2 × 38 mg), DMF | 33% II + |
| | (0.8 ml), 110° C., o/n | 1% XXV |
| 24-6 | XVIIIa (57 mg), XXIVb (67 mg), tBuONa (2 × 38 mg), 1,4- | 38% II + |
| | dioxane - DMF (0.8 + 0.2 ml), 110° C., o/n | 2% XXV |
| 24-7 | XVIIIa (57 mg), XXIVc (87 mg), tBuONa (2 × 38 mg), DMF | 12% II + |
| | (0.8 ml), 110° C., o/n | 1% XXV |
| 24-8 | XVIIIa (57 mg), XXIVc (87 mg), tBuONa (2 × 38 mg), 1,4- | 27% II + |
| | dioxane - DMF (0.8 + 0.2 ml), 110° C., o/n | 1% XXV |
| 24-9 | XVIIIa (57 mg), XXIVc (75 mg), 60 w % NaH (24 mg), THF (1 | 13% XXV |
| | ml), rt to 64° C., o/n | |
| 24-10 | XVIIIa (57 mg), XXIVf (89 mg), 60 w % NaH (24 mg), THF (1 | 7% II + |
| | ml), rt to 65° C., o/n | 27% XXV |
| 24-11 | XXIII (550 mg), XXIVb (770 mg), tBuOK (617 mg), NMP (20 | 12% XXV |
| | ml), 80° C., o/n | |
| 24-12 | XXIII (610 mg), XXIVb (859 mg), tBuOK (685 mg), tBuOH | 19% XXV |
| | (23 ml), reflux, 2 days | |
| 24-13 | XXIII (1.2 g), XXIVb (1.68 g), tBuONa (1.35 g), 15-crown-5 | 12% XXV |
| | (2.06 g), tBuOH (23 ml), reflux, o/n | |

### Example 25

Simeprevir (or a salt thereof) is prepared by preparing an intermediate using any of the processes steps described in Examples 1 to 24, following by conversion to Simeprevir (or a salt thereof, e.g. a sodium salt).

### Example 26

A pharmaceutical composition is prepared by first preparing Simeprevir (or a salt thereof), and then contacting Simeprevir (or a salt thereof) so obtained with a pharmaceutically acceptable carrier, diluent and/or excipient.

The invention may be described with respect to the following clauses.

Clause 1. A process for preparing a compound of formula (I)

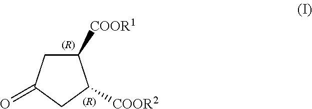

wherein
R¹ represents hydrogen or an alkyl group (for example a C₁₋₆ alkyl group, especially methyl);
R² represents an alkyl group (for example a C₁₋₆ alkyl group, especially methyl);
the two chiral centres are of an (R) configuration (thereby representing an enantioenriched form);
which comprises selective hydrolysis of a (trans-racemic) compound of formula (II)

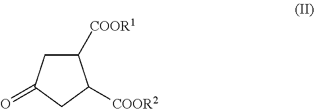

but in which
R¹ and R² independently represent an alkyl group (for example a C₁₋₆ alkyl group, especially methyl).
Clause 2. A process as claimed in Clause 1, where the selective hydrolysis is performed in the presence of an enzyme.
Clause 3. A process as claimed in Clause 2, wherein the enzyme is of the hydrolase class (e.g. a lipase).
Clause 4. A compound of formula (I) in enantioenriched form.
Clause 5. A process for the preparation of a compound of formula (I) as claimed in any of Clauses 1 to 3, further comprising any of the following conversion steps:
(i) Compounds of formula (I) in which R¹ is H converted to compounds of formula (I) in which R¹ is alkyl;
(ii) Compounds of formula (I) in which R¹ is H converted by undergoing a standard amide-coupling reaction in accordance with the following scheme:

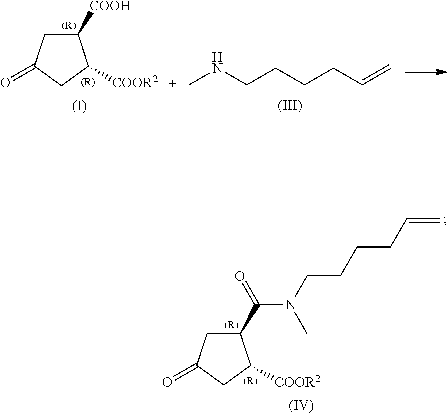

(iii) Compounds of formula (I) converted by undergoing a diastereoselective reduction reaction in accordance with the following scheme:

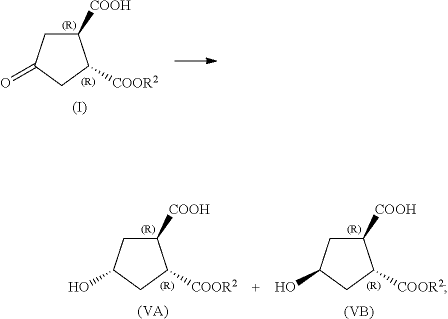

(iv) Compounds of formula (IV) converted by undergoing a diastereoselective reduction reaction in accordance with the following scheme:

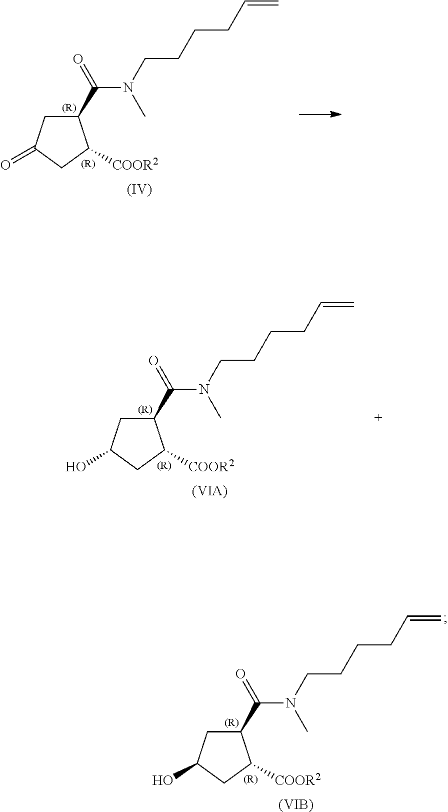

(v) Compounds of formula (VA) and (VB) may be converted to compounds of formula (VIA) and (VIB), respectively, for example under the conditions mentioned herein at (ii) above, i.e. an amide-coupling reaction in the presence of an amine of formula (III) and under the appropriate reaction conditions;
(vi) Compounds of formula (I) in which R¹ and R² both represent alkyl may also be converted, by a ketone reduction reaction, in accordance with the following scheme:

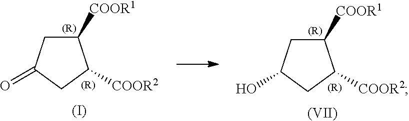

(vii) Compounds of formula (VII) converted by undergoing a selective hydrolysis reaction to form compounds of formula (VA) and (VB);
(viii) Compounds of formula (VIA) converted to compounds of formula (IX) by reaction with intermediate (VIII) in accordance with the scheme below:

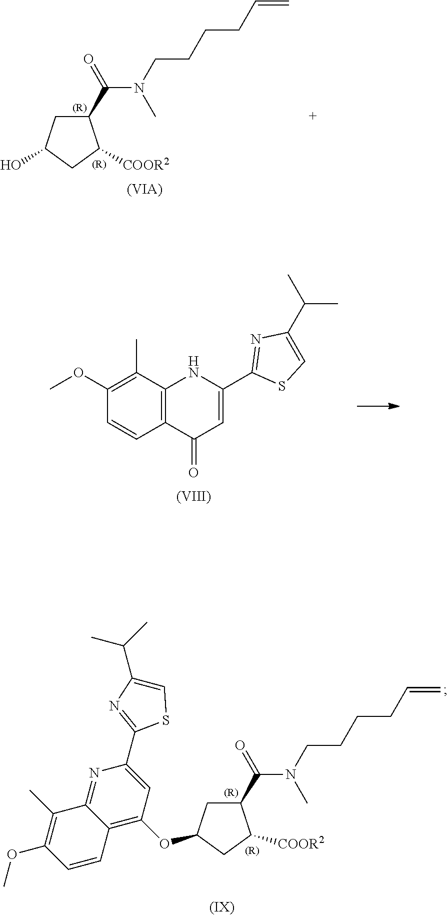

(ix) Compounds of formula (VIB) converted to compounds of formula (IX) (by reaction with intermediate (VIIIA)) in accordance with the scheme below:

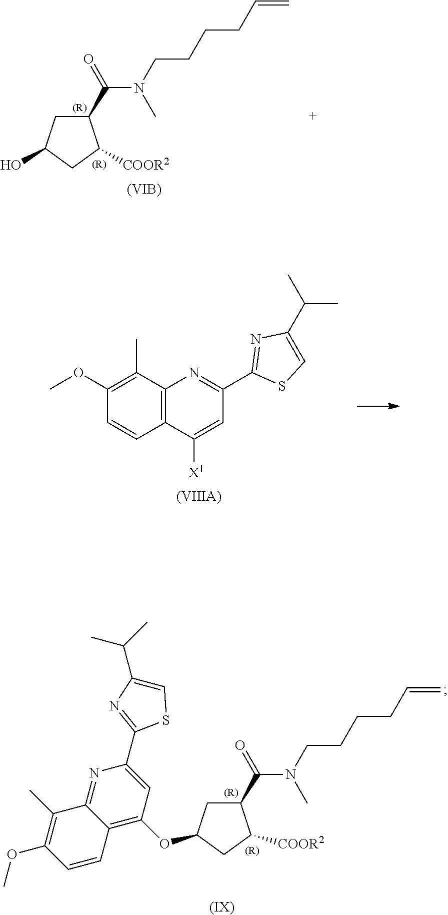

where R² is as defined in clause 1 or represents hydrogen;
(x) Compounds of formula (VIIIA) converted from compounds of formula (VIII).
   Clause 6. A compound of formula (IV) in enantioenriched form.
   Clause 7. A compound of formula (VA) and/or (VB), each of which are in enantioenriched form.
   Clause 8. A compound of formula (VIB) in enantioenriched form;
   Clause 9. A compound of formula (VII) in enantioenriched form.
   Clause 10. A process for the preparation of Simeprevir which comprises any of the process steps as claimed in Clauses 1 to 3 or 5, following by further conversion steps.
   Clause 11. A process for the preparation of Simeprevir as claimed in Clause 10 comprising a process as claimed in any one of Clauses 1 to 3, followed by further conversion steps as described in Clause 5, particularly process step (ii) followed by (iv) which in turn is followed by (viii) or (ix).
   Clause 12. A process as claimed in Clause 10 or Clause 11, wherein the further conversion steps include the following:

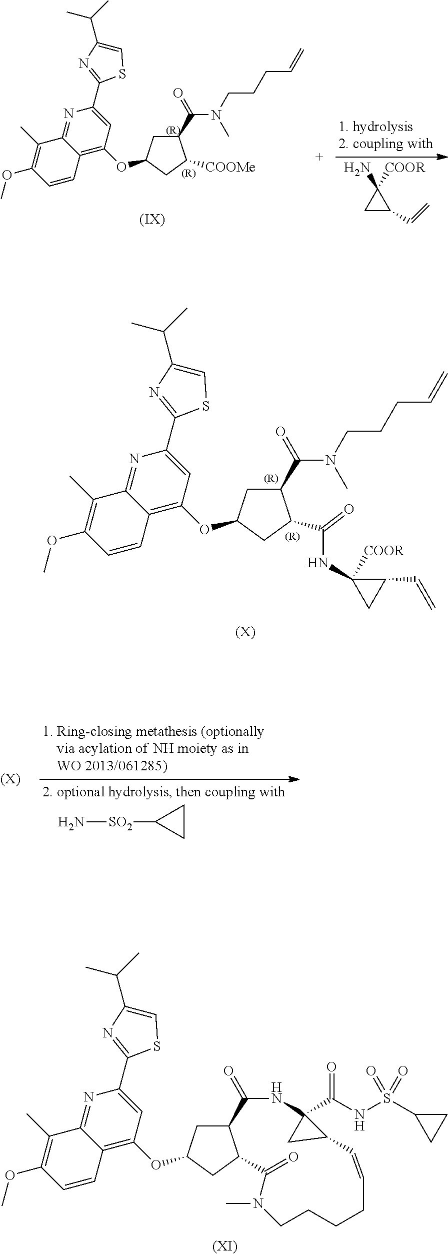

Clause 13. A pharmaceutical composition comprising Simeprevir (or a salt thereof) as obtained by any of Clauses 10, 11 or 12 (i.e. following such process steps).
Clause 14. A process for preparing a pharmaceutical composition as claimed in Clause 13, which comprises a process for preparing Simeprevir (or a salt thereof) as claimed by any of Clauses 10, 11 or 12, followed by contacting it with a pharmaceutically acceptable carrier, diluent and/or excipient.

1. A process for preparing a compound of formula (I) 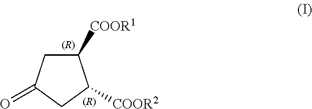
wherein
R¹ represents hydrogen or an alkyl group;
R² represents an alkyl group;
the two chiral centers are of an (R) configuration (thereby representing an enantioenriched form);
which comprises selective hydrolysis of a (trans-racemic) compound of formula (II) 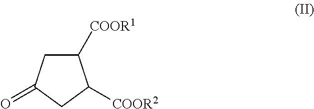
but in which
R¹ and R² independently represent an alkyl group, followed by:
(i) for compounds of formula (I) in which R¹ is H, an amide-coupling reaction in accordance with the following scheme: 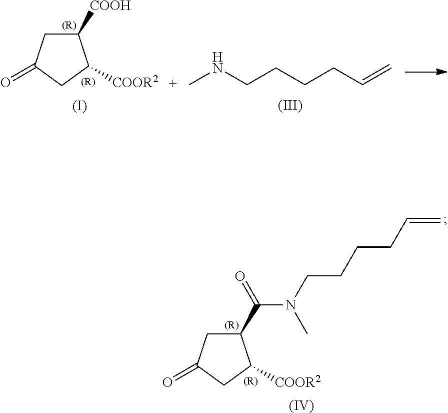
(ii) for compound of formula (I) in which R¹ is H, a diastereoselective reduction reaction in accordance with the following scheme: 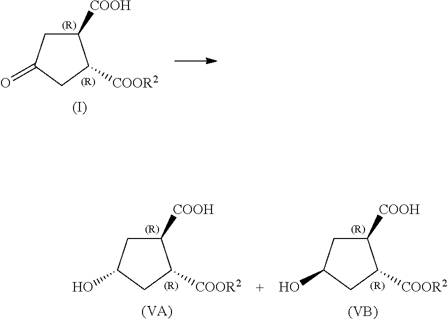
(iii) for compounds of formula (I) in which R¹ and R² both represent alkyl a ketone reduction reaction, in accordance with the following scheme:

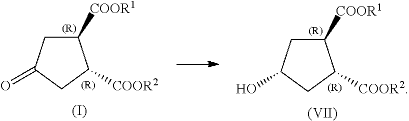

2. A process as claimed in claim 1, where the selective hydrolysis is performed in the presence of an enzyme.

3. A process as claimed in claim 2, wherein the enzyme is of the hydrolase class (e.g. a lipase).

4. A process for the preparation of a compound of formula (I) as claimed in claim 1, further comprising any of the following conversion steps:
(iv) Compounds of formula (IV) converted by undergoing a diastereoselective reduction reaction in accordance with the following scheme: 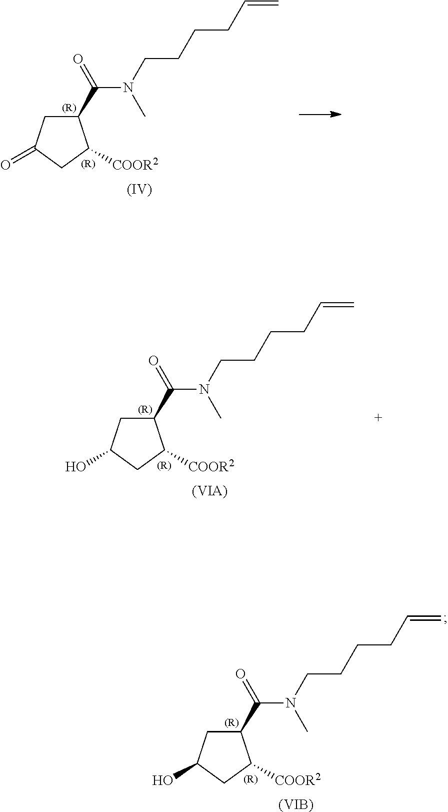
(v) Compounds of formula (VA) and (VB) converted to compounds of formula (VIA) and (VIB), respectively;
(vi) Compounds of formula (VII) converted by undergoing a selective hydrolysis reaction to form compounds of formula (VA) and (VB);
(vii) Compounds of formula (VIA) converted to compounds of formula (IX) by reaction with intermediate (VIII) in accordance with the scheme below: 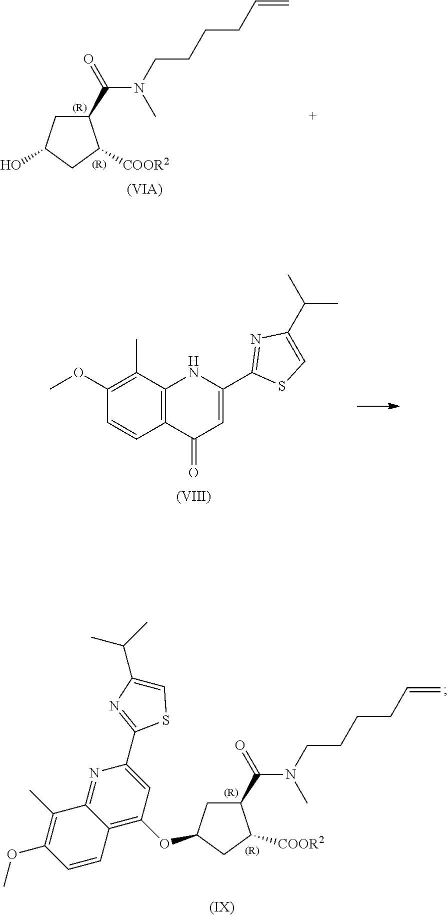
(viii) Compounds of formula (VIB) converted to compounds of formula (IX) (by reaction with intermediate (VIIIA)) in accordance with the scheme below: 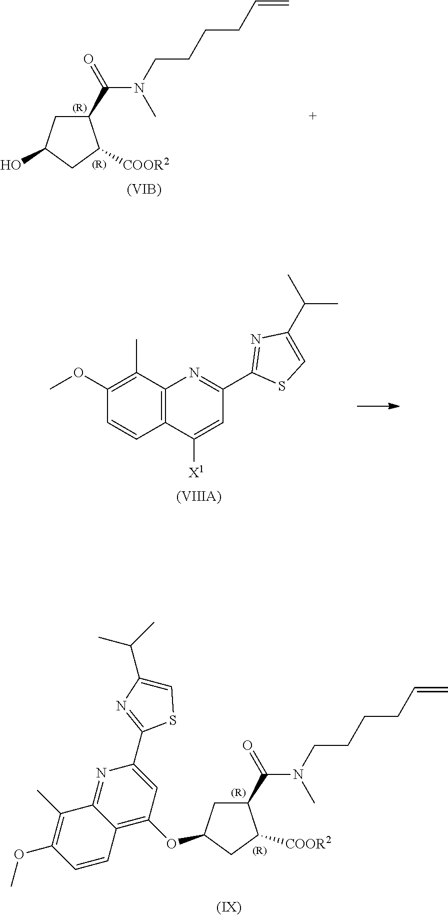
where R² is as defined in claim 1 or represents hydrogen;
(ix) Compounds of formula (VIIIA) converted from compounds of formula (VIII).

5. A compound selected from the group consisting of the compound of formula (IV), (VA), (VB), (VIB) or (VV), wherein said compound is in enantioenriched form.

6-10. (canceled)

11. The process of claim 1, further comprising the further conversion steps:

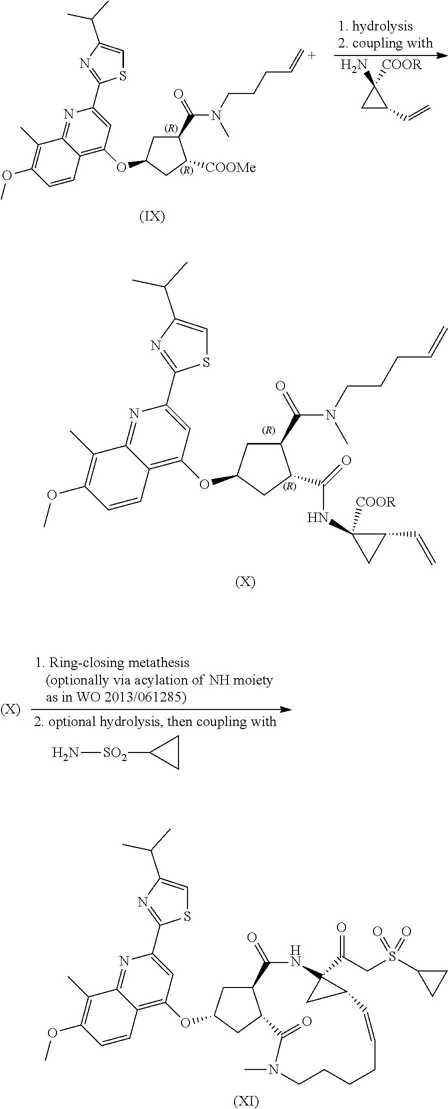

12-13. (canceled)
